SEQUENCE LISTING (59 sequences)

SEQ ID NO: 1 - protein, 261 aa, Anaplasma marginale
MSFKIKDERLSAHIANPDGTRYMRQGLGVTTALSVIVVAALAARVWRLSQEGLESSVLLKRPEFIALMVMLAVFVVAAIGLAVVCNEHQKKAKEMNGLYPNAAYLVAPARGAEGELGRRHETRHVSFVSSGRTNPFSTPTSMAGLVTLTVFAALSGALPSSTAITADLIGSGFAAATPLQQAWVVMLFLAMAVTFFAAMRGGLLASGQGNRLCVVSSGDVSAADGVLPAGALGPETDFNEVMAIHVHDANYRGGAATPVAM

SEQ ID NO: 2 - protein, 422 aa, Anaplasma marginale
VRVARVSFGLLRCDGFFKLGGVVLRSLVGATLAVLLPAVFLYGTGSSAAEAFGPYVSFGYTPAWGGVRNLYVGIPGETWYVLPYKKDVSGDEVLSWSSFDWWGKNGGAPGDDPIKFKRISPYGVTGAVGYALGDTRIELGVIGQEFSVSEISGRHWKQGNSLFLLLGKRSADLVRWLRPYISTNAGDGKSVEEGKRLNNLLLALRRGLNGLSESGRKAEAASAKMLLNYVGSATMPGSNWSSKPDVVKRLHTMLGQALPKVWPYLSYSDKDEAWRALGEYGDNGVVAISAVELTAVTVVGCRDLALSNLFTAAATRNLDAYGCAGMGVNFVRGAGKNVAEFGAELKLGVSYRLSRAASVFVGGVLHRTANYDFNLPVIPMGADSGSVAAAGGHADYARNEEARISFGVLNLAGEVGLRLILT

SEQ ID NO: 3 - protein, 330 aa, Anaplasma marginale
MKKVYGLVYAALSLLFTPCGSFASPRPIDFSRGEGASGFFASVQYKLAVPHFRDFIVEDKGKALNTFAMKEKQQGGTAKAAAGAATPPAASSDAEAPPAKGPDLASGGSFEGKYSPEYLRSAKAGSVSVGYSAGNVRLEAEGMYQKFPVDTKKYKDNPERAYRFAISAPDENSTTVATRPQEPYHITAENKEVTTASLMANLCYDLLPESSQISPSACVGGGGSLVRFLGVTEVRWAYQAKVGVQYFASRKAALFAYAYASRVHPEKFSNIPVVHHIKTESPKGSQGAAGSSGGESSAQAAGGKLPGLLYPQASLGLDYFGFECGIRLVL

SEQ ID NO: 4 - protein, 297 aa, Anaplasma marginale
MSRKSLFVLPCLLFVAVSTAGNGAAPNVGSAAPGVGAEGELYVAAQYKPALPVVREFAVRENRLTAPSKLFRLAPSTSVLTAEQATGATLLDSPLLRALRDRNNFEPSYTPSYEVSMCGVSGVLGYSRAGTRVELEVSFEDFRVKKSGKPVLKGGHEYFAAGRTSDTQRVVFANRAISAGSAVVSICRDFPAGPSGGSVTPYTCLGGGVEFLDILGMANTRFAYQAKMGAALNLHPRASIFAAGYYRGTLERAIRPLPAVVVIPATAGERENLGLPPFEGNAGVRYLGVEAGIRVLL

SEQ ID NO: 5 - DNA, 786 bp, Anaplasma marginale
atgtccttcaagattaaagatgagaggctgagtgcgcacatagcgaacccagatggtacgaggtatatgcgtcagggtttgggtgttactacagctttgtcggtcatagtagttgccgcgcttgcagcgcgtgtgtggaggctgtctcaggagggcctggaatctagcgtgcttctgaagcgtcccgagttcatagcgctgatggtcatgctggctgtgtttgttgttgctgctataggcttggccgtggtctgcaatgaacatcagaagaaggctaaggagatgaacggcctatatccgaatgccgcatacttggtagctcctgctaggggggcagaaggtgagctgggacggagacatgagacgcggcacgtgtcttttgtttcgagcggtcgcacaaatccgttctccacgcctacatctatggcgggtctggtgactctgactgtgttcgctgcgctgtctggtgcgctgcctagttctacggctattactgccgatttgataggttccgggtttgctgctgctactcctctgcaacaagcgtgggttgtcatgctattcttagctatggctgtaacgttctttgctgccatgagaggtggattgcttgcctccggtcaggggaacaggctttgtgttgtgagctctggtgatgtctccgctgcggatggggtcctccccgctggtgcgctgggtccagagaccgacttcaacgaggttatggctatacacgtacacgatgctaattacaggggcggcgcggcaactcccgttgcaatgtaa

SEQ ID NO: 6 - DNA, 1269 bp, Anaplasma marginale
gtgagggtcgcacgcgtgtccttcgggttgctgcgttgcgacggtttctttaagctcggaggggttgtgctcaggtctctcgtaggtgccactctggcagtgttgctgcctgctgtctttctttacggaacaggttcttctgctgccgaagcatttggtccgtacgtaagtttcggatatacaccagcttggggaggtgttagaaacctatacgttggcattcctggagagacttggtatgtcctgccctataagaaagatgtatcaggtgatgaagtattatcctggtctagctttgactggtggggtaagaacggaggcgctcctggtgatgatccaataaagtttaaacgtataagcccatatggtgtcactggagctgtaggctatgctctgggtgacactaggatagagcttggagtgattgggcaagagttttctgtttctgagattagtggtagacattggaaacaaggaaactccttgttcttgttgctagggaagcgtagtgcagatcttgtgaggtggttgcgcccctacattagtactaatgccggcgatggtaagtccgtggaagaaggtaagaggcttaacaatcttctgctcgcactccgccgtggcctgaatgggctatctgaatccggacggaaggcagaagcagcaagcgcgaagatgttgcttaactatgttgggtcggcgaccatgcccggcagcaactggtcaagtaagccggatgttgtcaaacggctacacaccatgctcgggcaggcgttgcctaaggtctggccatatctttcatatagcgataaggacgaagcgtggcgtgccctgggtgagtatggggacaacggggttgtggcgatttctgctgttgagctcaccgctgttacggttgttgggtgtcgcgatcttgcgctgtcgaatctatttacggccgcggctactcggaacttggatgcgtacggttgtgctggaatgggggtaaactttgtacgcggggccgggaagaatgttgcggaatttggtgctgagttgaagcttggtgtgagctacagattatctcgcgctgcatcggtgtttgttggcggggtgctgcatagaactgcgaattatgattttaacttgcccgttattcctatgggggctgatagtgggtctgtggcggcggcgggaggccatgcagactacgcaaggaatgaggaggctcggatctcttttggggtgctgaatctcgctggagaagtgggcctgaggcttatactcacgtag

SEQ ID NO: 7 - DNA, 993 bp, Anaplasma marginale
atgaagaaggtctacggcttggtttacgctgcgttgtctttgttgttcacgccttgcgggtcatttgcttcccccaggcccatagacttctctaggggcgagggtgcgtcaggattcttcgccagtgtacagtacaagctggcagtcccgcactttagggactttatagtagaagacaaaggtaaggcgcttaatacgtttgccatgaaggaaaaacagcagggtggcaccgctaaggcagcggcgggcgctgcaacgcctccagcggcctcgtccgacgccgaggctccgcccgctaaagggccagatctagcttcgggcggcagctttgaggggaagtactcccctgagtaccttaggagtgctaaggccgggtccgtatcggttgggtactccgctggaaatgtgcggctggaggctgaaggcatgtatcagaaattccccgtagatactaagaagtacaaggacaatcctgagcgcgcgtatcgttttgccatttcagcgcctgatgagaatagtacaactgtcgctacaaggccgcaggagccctaccacatcaccgctgagaataaggaagtgaccacagcatccctgatggccaatctgtgctacgacctcttgcctgagtcctcccagatctctccaagcgcctgcgtcggcggtggtggcagtctagtcaggtttctgggcgtgaccgaggtgcggtgggcgtaccaggcaaaggttggggtgcagtatttcgcgtctaggaaggcagccctgttcgcgtacgcttatgcaagtagggtgcatccggaaaagttctccaacatacctgtagtacaccacatcaagacagagtcacctaaaggatcccagggtgcagcaggtagcagtgggggcgagagcagcgcccaggcggccggcggtaagctgcccggtttgctatacccgcaagcgagcttggggctggactattttgggtttgagtgcgggataagactcgtactttag

SEQ ID NO: 8 - DNA, 894 bp, Anaplasma marginale
atgagtcgtaaaagtctgttcgtgctgccttgtcttctctttgtggcggtgtccaccgccgggaacggggcggctcccaatgttggtagcgccgcaccgggcgtcggggcggagggtgagctctatgtagcggctcagtacaagccggcactgcctgtggtgcgggagttcgccgtgcgtgaaaacaggcttaccgctccaagcaagctttttaggctcgcgcccagcacatctgttctgactgccgagcaggcaacgggtgctacactcttagactctcctctgctcagggctctgcgggataggaataactttgagcctagctacacgccatcatacgaggtcagtatgtgtggggtttcaggtgtgctgggctactccagagcgggcaccagagtggaacttgaggtttcttttgaggatttcagggttaagaagtccgggaagccggtgctcaaaggcgggcacgaatattttgccgctgggagaaccagcgacactcaacgggttgtttttgcaaatcgcgcaatctctgctggctcagccgtagttagcatctgtcgcgatttccctgcaggcccctctggaggaagcgttaccccgtacacatgcctgggcggcggcgtggagttccttgacattcttggcatggcgaacacacgcttcgcataccaggcgaaaatgggcgcagctttaaacttgcaccctcgtgccagcatcttcgcagcaggttactaccggggaaccctggagcgggctattaggccccttcctgccgtggtcgtgataccggctactgctggggagagggagaacttgggcctgccaccgttcgagggtaacgcaggagtgcggtaccttggcgtcgaagcaggcataagggtgttactttga

SEQ ID NO: 9 - protein, 16 aa, Anaplasma marginale
CQRVAAQERSRELSRA

SEQ ID NO: 10 - protein, 15 aa, Anaplasma marginale
CVDGHINPKFAYRVK

SEQ ID NO: 11 - protein, 21 aa, Anaplasma marginale
ESSKETSYVRGYDKSIATIDC

SEQ ID NO: 12 - protein, 25 aa, Anaplasma marginale
SFKIKDERLSAHIANPDGTRYMRQG

SEQ ID NO: 13 - protein, 9 aa, Anaplasma marginale
  misc_feature (6)..(6): X = S or R
HVSFVXSGR

SEQ ID NO: 14 - protein, 16 aa, Anaplasma marginale
  misc_feature (1)..(1): X = E or G
XMXGLYPNAAYLVAPA

SEQ ID NO: 15 - protein, 18 aa, Anaplasma marginale
AAPNVGSAAPGVGAEGEL

SEQ ID NO: 16 - protein, 23 aa, Anaplasma marginale
  misc_feature (8)..(8): X = S or Q
SPRPIDEXRGEGASGFFASVQYK

SEQ ID NO: 17 - protein, 9 aa, Anaplasma marginale
VGVQYFASR

SEQ ID NO: 18 - protein, 11 aa, Anaplasma marginale
  misc_feature (3)..(3): X = L or I
AAXFAYAYASR

SEQ ID NO: 19 - protein, 13 aa, Anaplasma marginale
  misc_feature (4)..(4): X = L or I
GPDXASGGSFEGK

SEQ ID NO: 20 - protein, 12 aa, Anaplasma marginale
  misc_feature (10)..(10): X = L or I
QSVSVGYSEXVR

SEQ ID NO: 21 - protein, 19 aa, Anaplasma marginale
AEAFGPYVSFGYTPAAGDV

SEQ ID NO: 22 - protein, 10 aa, Anaplasma marginale
  misc_feature (4)..(4): X = any amino acid
LNLXXLFTAT

SEQ ID NO: 23 - protein, 8 aa, Anaplasma marginale
PYLSYSDK

SEQ ID NO: 24 - protein, 10 aa, Anaplasma marginale
HTMLGQALPK

SEQ ID NO: 25 - protein, 11 aa, Anaplasma marginale
ASVFVGGVLHR

SEQ ID NO: 26 - protein, 10 aa, Anaplasma marginale
  misc_feature (1)..(1): X = I or L
XXPYGVTGAV

SEQ ID NO: 27 - DNA, 18 bp, Artificial sequence
  Degenerate PCR primer
gacggnacnagrtayatg

SEQ ID NO: 28 - DNA, 20 bp, Artificial sequence
  Degenerate PCR primer
ggatanarnccytccatytc

SEQ ID NO: 29 - DNA, 19 bp, Artificial sequence
  Degenerate PCR primer
cgctwgcgtawgcrtangc

SEQ ID NO: 30 - DNA, 23 bp, Artificial sequence
  Degenerate PCR primer
ttcttcgcwagygtncartayaa

SEQ ID NO: 31 - DNA, 23 bp, Artificial sequence
  Degenerate PCR primer
gctgargcnttyggnccntaygt

SEQ ID NO: 32 - DNA, 23 bp, Artificial sequence
  Degenerate PCR primer
actgcbccwgtnacnccrtangg

SEQ ID NO: 33 - DNA, 26 bp, Anaplasma marginale
tgatgaagtattatcctggtctagct

SEQ ID NO: 34 - DNA, 20 bp, Artificial sequence
  Degenerate PCR primer
gcytgnccnagcatngtrtg

SEQ ID NO: 35 - DNA, 20 bp, Anaplasma marginale
tagcgctgatggtcatgctg

SEQ ID NO: 36 - DNA, 22 bp, Anaplasma marginale
cgtggtctgcaatgaacatcag

SEQ ID NO: 37 - DNA, 24 bp, Anaplasma marginale
tcggacgagcccgctggaggcgtt

SEQ ID NO: 38 - DNA, 26 bp, Anaplasma marginale
aagcgccttacctttgtcttctacta

SEQ ID NO: 39 - DNA, 25 bp, Anaplasma marginale
caggacataccaagtctctccagga

SEQ ID NO: 40 - DNA, 24 bp, Anaplasma marginale
caacgtataggtttctaacacctc

SEQ ID NO: 41 - DNA, 20 bp, Anaplasma marginale
ctgacgcatatacctcgtac

SEQ ID NO: 42 - DNA, 19 bp, Anaplasma marginale
ggcaactactatgaccgac

SEQ ID NO: 43 - DNA, 24 bp, Anaplasma marginale
gcggtggtggcagtctagtcaggt

SEQ ID NO: 44 - DNA, 24 bp, Anaplasma marginale
ggtgcagtatttcgcgtctaggaa

SEQ ID NO: 45 - DNA, 25 bp, Anaplasma marginale
taactatgttgggtcggcgaccatg

SEQ ID NO: 46 - DNA, 24 bp, Anaplasma marginale
ccggcagcaactggtcaagtaagc

SEQ ID NO: 47 - DNA, 24 bp, Artificial sequence
  PCR primer
gaggatccatgtccttcaagatta

SEQ ID NO: 48 - DNA, 27 bp, Artificial sequence
  PCR primer
tacccgggttacattgcaacgggagtt

SEQ ID NO: 49 - DNA, 22 bp, Artificial sequence
  PCR primer
tggatccggcggctcccaatgt

SEQ ID NO: 50 - DNA, 26 bp, Artificial sequence
  PCR primer
acccgggtcaaagtaacacccttatg

SEQ ID NO: 51 - DNA, 25 bp, Artificial sequence
  PCR primer
gcatgctcccccaggcccatagact

SEQ ID NO: 52 - DNA, 27 bp, Artificial sequence
  PCR primer
cccgggataagtacgagtcttatnccg

SEQ ID NO: 53 - DNA, 27 bp, Artificial sequence
  PCR primer
ggatccgccgaagcatttggtccgtac

SEQ ID NO: 54 - DNA, 26 bp, Artificial sequence
  PCR primer
gtcgacctacgtgagtataagcctca

SEQ ID NO: 55 - DNA, 25 bp, Artificial sequence
  PCR primer
tggatccatgagtcgtaaaagtctg

SEQ ID NO: 56 - DNA, 894 bp, Anaplasma centrale
atgagtcgtaaaagtctgttcgtgctgccttgtcttctctttgtggcggtgtccaccgccgggaacggggcggctcccaatgttggtagcgccgcaccgggcgtcggggcggagggtgagctctatgtagcggctcagtacaagccggcactgcctgtggtgcgggagttcgccgtgcgtgaaaacaggcttaccgctccaagcaagctttttaggctcgcgcccagcacatctgttctgactgccgagcaggcaacgggtgctacactcttagactctcctctgctcagggctctgcgggataggaataactttgagcctagctacacgccatcatacgaggtcagtatgtgtggggtttcaggtgtgctgggctactccagagcgggcaccagagtggaacttgaggtttcttttgaggatttcagggttaagaagtccgggaagccggtgctcaaaggcgggcacgaatattttgccgctgggagaaccagcgacactcagcgggttgtttttgcaaatcgcgcaatctctgctggctcagccgtagttagcatctgtcgcgatttccctgcaggcccctctggaggaagcgttaccccgtacacatgcctgggcggcggcgtggagttccttgacattcttggcatggcgaacacacgcttcgcataccaggcgaaaatgggcgcagctttaaacttgcaccctcgtgccagcatcttcgcagcaggttactaccggggaaccctggagcgggctattaggccccttcctgccgtggtcgtgataccggctactgctggggagagggagaacttgggcctgccaccgttcgagggtaacgcaggagtgcggtaccttggcgtcgaagcaggcataagggtgttactttga

SEQ ID NO: 57 - DNA, 783 bp, Anaplasma centrale
atgtccttcaagattaaagatgagaggctgagtgcgcacatagcgaacccagatggtacgaggtatatgcgtcagggtttgggtgttactacagctttgtcggtcatagtagttgccgcgcttgcagcgcgtgtgtggaggctgtctcaggagggcctggaatctagcgtgcttctgaagcgtcccgagttcatagcgctgatggtcatgctggctgtgtttgttgttgctgctataggcttggccgtggtctgcaatgaacatcagaagaaggctaaggagatgaacggcctgtatccgaatgccgcatacttggtagctcctgctaggggggcagaaggtgagctgggacggagacatgagacgcggcacgtgtcttttgtttcgagcggtcgcacaaatccgttctccacgcctacatctatggcgggtctggtgactctgactgtgttcgctgcgctgtctggtgcgctgcctagttctacggctattactgccgatttgataggttccgggtttgctgctgctactcctctgcaacaagcgtgggttgtcatgctattcttagctatggctgtaacgttctttgctgccatgagaggtggattgcttgcctccggtcaggggaacaggctttgtgttgtgagctctggtgatgtctccgctgcagatggggtcctccccgctggtgcgctgggtccagagaccgacttcaacgaggttatggctatacacgtacacgatgctaattacaggggcggcgcggcaactcccgttgcaatg

SEQ ID NO: 58 - protein, 20 aa, Anaplasma marginale
RLSQEGLESSVLLKRPEFIA

SEQ ID NO: 59 - protein, 20 aa, Anaplasma marginale
TADLIGSGFAAATPLQQAWV

(19) United States
(12) Patent Application Publication
(10) Pub. No.: US 2006/0088546 A1
(43) Pub. Date: Apr. 27, 2006

(54) Antigens for raising an immune response against rickettsieae and ehrlichieae pathogens

(76) Inventors: George Alfred Riding, Yeronga (AU); Michelle Anne Hope, Acacia Ridge (AU); Peter Willadsen, Chapel Hill (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/474,853

(22) PCT Filed: Apr. 11, 2002
(86) PCT No.: PCT/AU02/00468

§ 371 Date: May 3, 2004

(30) Foreign Application Priority Data
Apr. 12, 2001 (AU) PR 4400
Sep. 10, 2001 (AU) PR 7597
Mar. 1, 2002 (AU) PS 0861

(51) Int. Cl. A61K 48/00; C07H 21/04; A61K 39/02; C07K 14/195; A61K 36/18; A01H 1/00; C12N 15/74

(52) U.S. Cl. 424/190.1; 514/44; 800/288; 530/388.4; 530/350; 435/69.3; 435/320.1; 435/252.3; 536/23.7

(57) ABSTRACT

The present invention relates to the identification of polypeptides which are useful for raising an immune response against Ehrlichieae and Rickettsieae pathogens when administered into a subject. These polypeptides, and polynucleotides encoding therefor, can be used in various strategies for preventing or treating Ehrlichieae and Rickettsieae infections.

### FIELD OF THE INVENTION

The present invention relates to the identification of polypeptides which are useful for raising an immune response against Ehrlichieae and Rickettsieae pathogens when administered into a subject. More particularly, the present invention relates to the identification of antigenic polypeptides from Anaplasma marginale.

### BACKGROUND OF THE INVENTION

Tick-borne diseases are a major problem of domestic livestock production in large areas of the world, though they are at their most acute in tropical and subtropical regions. The causative agents of tick fevers include Babesia bovis, Anaplasma marginale and Babesia bigemina, the first two being the most important and pathogenic.

Anaplasma marginale, the major pathogen causing anaplasmosis, is an intra-erythrocytic rickettsia, which causes extra-vascular erythrocyte destruction. Pathology is manifested as an anaemia that, especially in non-immune adult cattle, causes serious morbidity and often mortality. Cattle that survive initial infection, although carriers of the pathogen, are resistant to clinical disease.

Live attenuated vaccines for these diseases have been produced. A degree of immunity against Anaplasma marginale is conferred by vaccination of cattle with bovine blood infected with the less virulent Anaplasma centrale. However, like many such attenuated vaccines they have disadvantages. A significant level of pathology is induced on vaccination and in some animals, particularly older cattle, this can be severe. Secondly, despite careful quality control, there is always a significant risk that other disease organisms can be inadvertently transmitted with the vaccine. Thirdly, particularly in the case of Babesia bovis, there is a possibility of reversion to virulence following transmission by ticks of the partially attenuated vaccine organisms. This leads, at the very least, to a reluctance to use the vaccine in areas where disease incidence is not high while in some areas the vaccine cannot be used at all.

Low coverage is also due, in part, to the use of Bos indicus cattle which are much more resistant to ticks and babesiosis than the more productive Bos taurus breeds. However, a recent study (Bock et al. 1997) has shown that Bos taurus and Bos indicus breeds are equally susceptible to anaplasmosis, thus supporting the use of a stand-alone non-living vaccine against A. marginale. Further, in some areas, the move towards ‘finishing’ cattle in feedlots is also significant for the future use of vaccines against A. marginale as under intensive conditions mechanical transmission is far more likely to occur.

In the U.S. where the use of live A. centrale is precluded, A. marginale has been purified from infected blood on a commercial scale and used as a non-living vaccine (e.g. ‘Anaplaz’, Fort Dodge Laboratories, ‘Am-Vax’, Scheering Plough, ‘Plaz-Vax’, Mallinckrodt). Safety problems with these vaccines have occurred on several occasions in the past due to contamination of the product with host red cell antigens which results in mortality due to isoerythrolysis in suckling calves of vaccinated mothers.

This same complex of diseases is of even greater importance over large areas of Central and South America and in parts of East Asia, for example the southern half of China. In most of these areas attenuated live vaccines are not available or their use is very restricted due, in part, to their relatively high cost. The great disincentive, however, is the difficulty of maintaining adequate quality control of the vaccine and ensuring its delivery in effective form in all areas.

Despite the fact that protective vaccination with killed Anaplasma material has been repeatedly demonstrated (Montenegro-James et al., 1991) little is known of the protective antigens themselves. A major difficulty in identifying novel antigens is the presence of large amounts of immunodominant but poorly protective, known antigens which are, in most cases, highly variable. Work has focussed on a complex mixture of surface proteins labelled the Major Surface Protein complex (MSP) (e.g. Vidotto et al., 1994) and in particular on a neutralization-sensitive surface protein Am105 (Palmer et al., 1986). These proteins have been available for some time, but progress in generating a recombinant vaccine has been slow. The available evidence suggests the antigens are of inadequate efficacy.

Anaplasma species which infect ruminants other than cattle include Anaplasma ovis which infects sheep. Closely related pathogens include; Cowdria ruminantium (also known as heartwater) which is a major problem in South Africa and the Carribean, numerous Ehrlichieae and Rickettsieae pathogens of domestic animals (including horses), as well as pathogens infecting humans such as Ehrlichia phagocytophila, Ehrlichia chaffeensis, Rickettsia prowazekii (which causes epidemic typhus), Rickettsia rickettsii and Rickettsia conorii (both of which cause spotted fever), and Ehrlichia sp. which cause human granulocytic ehrlichiosis.

The present inventors have now identified and characterized polypeptides which can be used in a vaccine to provide immune protection against Ehrlichieae and Rickettsieae pathogens.

### SUMMARY OF THE INVENTION

In one aspect, the present invention provides a vaccine comprising at least one polypeptide selected from the group consisting of:

a) a sequence provided in SEQ ID NO:1;

b) a polypeptide which is at least 50% identical to (a);

c) a sequence provided in SEQ ID NO:2;

d) a polypeptide which is at least 50% identical to (c);

e) a sequence provided in SEQ ID NO:3;

f) a polypeptide which is at least 50% identical to (e);

g) a sequence provided in SEQ ID NO:4; and

h) a polypeptide which is at least 50% identical to (g);

wherein the polypeptide raises an immune response against Ehrlichieae and/or Rickettsieae pathogens when administered to a subject.

Preferably, the polypeptide is at least 60% identical, more preferably at least 70% identical, more preferably at least 80% identical, more preferably at least 85% identical, more preferably at least 90% identical, more preferably at least 95% identical, and even more preferably at least 99% identical to (a), (c), (e) or (g).

Preferably, the polypeptide has an N-terminal sequence as provided in SEQ ID NO:12 and has a molecular weight of approximately 17 kDa.

Further, it is preferred that the polypeptide can be purified from a species of Ehrlichieae or Rickettsieae. Preferably, the polypeptide can be purified from a species selected from the group consisting of: Anaplasma sp., Ehrlichia sp., Rickettsia sp. and Cowdria sp. More preferably, the polypeptide can be purified from the group consisting of: Anaplasma marginale, Anaplasma centrale, Anaplasma ovis, Cowdria ruminantium, Ehrlichia equi, Ehrlichia phagocytophila, Ehrlichia chaffeensis, Rickettsia prowazekii, Rickettsia rickettsii, Rickettsia conorii, and Ehrlichia sp. which cause human granulocytic ehrlichiosis. Even more preferably, the polypeptide can be purified from Anaplasma marginale.

Preferably, the vaccine comprises a pharmaceutically acceptable carrier. It is also preferred that the vaccine comprises an adjuvant.

As is known in the art, an immune response can be provided through the use of DNA vaccines. Accordingly, in another aspect the present invention provides a DNA vaccine comprising at least one polynucleotide selected from the group consisting of:

a) a sequence encoding a polypeptide provided in SEQ ID NO:1;

b) a sequence encoding a polypeptide which is at least 50% identical to SEQ ID NO:1;

c) a sequence encoding a polypeptide provided in SEQ ID NO:2;

d) a sequence encoding a polypeptide which is at least 50% identical to SEQ ID NO:2;

e) a sequence encoding a polypeptide provided in SEQ ID NO:3;

f) a sequence encoding a polypeptide which is at least 50% identical to SEQ ID NO:3;

g) a sequence encoding a polypeptide provided in SEQ ID NO:4; and

h) a sequence encoding a polypeptide which is at least 50% identical to SEQ ID NO:4;

wherein the polypeptide encoded by the polynucleotide raises an immune response against Ehrlichieae and/or Rickettsieae pathogens when the DNA vaccine is administered to a subject.

Preferably, the polynucleotide encodes a polypeptide which is at least 60% identical, more preferably at least 70% identical, more preferably at least 80% identical, more preferably at least 85% identical, more preferably at least 90% identical, more preferably at least 95% identical, and even more preferably at least 99% identical to any one of SEQ ID NO's 1 to 4.

Preferably, the polynucleotide encodes a polypeptide which has an N-terminal sequence as provided in SEQ ID NO:12 and has a molecular weight of approximately 17 kDa.

Further, it is preferred that the polynucleotide can be isolated from a species of Ehrlichieae or Rickettsieae. Preferably, the polynucleotide can be isolated from a species selected from the group consisting of: Anaplasma sp., Ehrlichia sp., Rickettsia sp. and Cowdria sp. More preferably, the polynucleotide can be isolated from the group consisting of Anaplasma marginale, Anaplasma centrale, Anaplasma ovis, Cowdria ruminantium, Ehrlichia equi, Ehrlichia phagocytophila, Ehrlichia chaffeensis, Rickettsia prowazekii, Rickettsia rickettsii, Rickettsia conorii, and Ehrlichia sp. which cause human granulocytic ehrlichiosis. Even more preferably, the polynucleotide can be isolated from Anaplasma marginale.

In another preferred embodiment, the polynucleotide is contained in a vector. More preferably, the vector is a viral vector.

In a further aspect, the present invention provides a method for raising an immune response against an Ehrlichieae or Rickettsieae pathogen in a subject, the method comprising administering to the subject at least one vaccine according to the present invention.

In yet another aspect, the present invention provides a method of treating or preventing an Ehrlichieae or Rickettsieae infection in a subject, the method comprising administering to the subject at least one vaccine according to the present invention.

Preferably, the Ehrlichieae or Rickettsieae pathogen is selected from the group consisting of: Anaplasma sp., Ehrlichia sp., Rickettsia sp. and Cowdria sp. More preferably, the Ehrlichieae or Rickettsieae pathogen is selected from the group consisting of: Anaplasma marginale, Anaplasma centrale, Anaplasma ovis, Cowdria ruminantium, Ehrlichia equi, Ehrlichia phagocytophila, Ehrlichia chaffeensis, Rickettsia prowazekii, Rickettsia rickettsii, Rickettsia conorii, and Ehrlichia sp. which cause human granulocytic ehrlichiosis. Even more preferably, the Ehrlichieae or Rickettsieae pathogen is Anaplasma marginale.

Preferably, the subject is a mammal. In one embodiment, the mammal is selected from the group consisting of; cows, sheep, goats, dogs and horses. In another embodiment, the mammal is a human.

In a further aspect, the present invention provides for the use of a vaccine according to the present invention for the manufacture of a medicament for raising an immune response against an Ehrlichieae or Rickettsieae pathogen in a subject.

It is also known in the art that an immune response can be provided by the consumption of a transgenic plant expressing an antigen. Thus, in a further aspect the present invention provides a transgenic plant which produces at least one polypeptide selected from the group consisting of:

a) a sequence provided in SEQ ID NO:1;

b) a polypeptide which is at least 50% identical to (a);

c) a sequence provided in SEQ ID NO:2;

d) a polypeptide which is at least 50% identical to (c);

e) a sequence provided in SEQ ID NO:3;

f) a polypeptide which is at least 50% identical to (e);

g) a sequence provided in SEQ ID NO:4; and

h) a polypeptide which is at least 50% identical to (g);

wherein the polypeptide raises an immune response against Ehrlichieae and/or Rickettsieae pathogens when the transgenic plant is orally administered to a subject.

Preferably, the polypeptide is at least 60% identical, more preferably at least 70% identical, more preferably at least 80% identical, more preferably at least 85% identical, more preferably at least 90% identical, more preferably at least 95% identical, and even more preferably at least 99% identical to (a), (c), (e) or (g).

In yet another aspect, the present invention provides a method for raising an immune response against an Ehrlichieae or Rickettsieae pathogen in a subject, the method comprising orally administering to the subject at least one transgenic plant of the invention.

In a further aspect, the present invention provides a method of treating or preventing an Ehrlichieae or Rickettsieae infection in a subject, the method comprising orally administering to the subject at least one transgenic plant of the invention.

In another aspect, the present invention provides an antibody raised against a polypeptide selected from the group consisting of:

a) a sequence provided in SEQ ID NO:1;

b) a polypeptide which is at least 50% identical to (a);

c) a sequence provided in SEQ ID NO:2;

d) a polypeptide which is at least 50% identical to (c);

e) a sequence provided in SEQ ID NO:3;

f) a polypeptide which is at least 50% identical to (e);

g) a sequence provided in SEQ ID NO:4; and

h) a polypeptide which is at least 50% identical to (g);

wherein the antibody provides immune protection against Ehrlichieae and/or Rickettsieae pathogens when administered to a subject.

Preferably, the polypeptide is at least 60% identical, more preferably at least 70% identical, more preferably at least 80% identical, more preferably at least 85% identical, more preferably at least 90% identical, more preferably at least 95% identical, and even more preferably at least 99% identical to (a), (c), (e) or (g).

In a further aspect, the present invention provides a method of treating or preventing an Ehrlichieae or Rickettsieae infection in a subject, the method comprising administering to the subject at least one antibody according to the invention.

In another aspect, the present invention provides a substantially purified polypeptide which specifically binds to an antibody according to the invention.

In another aspect, the present invention provides a substantially purified polypeptide, the polypeptide being selected from:

(i) a polypeptide comprising the sequence provided as SEQ ID NO:1; and

(ii) a polypeptide which is at least 50% identical to (i);

wherein the polypeptide raises an immune response against Ehrlichieae and/or Rickettsieae pathogens when administered to a subject.

Preferably, the polypeptide is at least 60% identical, more preferably at least 70% identical, more preferably at least 80% identical, more preferably at least 85% identical, more preferably at least 90% identical, more preferably at least 95% identical, and even more preferably at least 99% identical to (i).

Preferably, the polypeptide has an N-terminal sequence as provided in SEQ ID NO:12 and has a molecular weight of approximately 17 kDa.

In another aspect, the present invention provides a substantially purified polypeptide, the polypeptide being selected from:

(i) a polypeptide comprising the sequence provided as SEQ ID NO:2; and

(ii) a polypeptide which is at least 50% identical to (i);

wherein the polypeptide raises an immune response against Ehrlichieae and/or Rickettsieae pathogens when administered to a subject.

Preferably, the polypeptide is at least 60% identical, more preferably at least 70% identical, more preferably at least 80% identical, more preferably at least 85% identical, more preferably at least 90% identical, more preferably at least 95% identical, and even more preferably at least 99% identical to (i).

In another aspect, the present invention provides a substantially purified polypeptide, the polypeptide being selected from:

(i) a polypeptide comprising the sequence provided as SEQ ID NO:3; and

(ii) a polypeptide which is at least 50% identical to (i);

wherein the polypeptide raises an immune response against Ehrlichieae and/or Rickettsieae pathogens when administered to a subject.

Preferably, the polypeptide is at least 60% identical, more preferably at least 70% identical, more preferably at least 80% identical, more preferably at least 85% identical, more preferably at least 90% identical, more preferably at least 95% identical, and even more preferably at least 99% identical to (i).

In another aspect, the present invention provides a substantially purified polypeptide, the polypeptide being selected from:

(i) a polypeptide comprising the sequence provided as SEQ ID NO:4; and

(ii) an antigenic fragment of (i),

wherein the polypeptide, or antigenic fragment thereof, raises an immune response against Ehrlichieae and/or Rickettsieae pathogens when administered to a subject.

Further, it is preferred that the polypeptide can be purified from a species of Ehrlichieae or Rickettsieae. Preferably, the polypeptide can be purified from a species selected from the group consisting of: Anaplasma sp., Ehrlichia sp., Rickettsia sp. and Cowdria sp. More preferably, polypeptide can be purified from the group consisting of: Anaplasma marginale, Anaplasma centrale, Anaplasma ovis, Cowdria ruminantium, Ehrlichia equi, Ehrlichia phagocytophila, Ehrlichia chaffeensis, Rickettsia prowazekii, Rickettsia rickettsii, Rickettsia conorii, and Ehrlichia sp. which cause human granulocytic ehrlichiosis. Even more preferably, the polypeptide can be purified from Anaplasma marginale.

It is preferred that the immune response is against Anaplasma marginale infection.

In a further preferred embodiment, a polypeptide of the present invention is obtainable by

(i) disrupting Anaplasma marginale in a sample to obtain a homogenate,

(ii) centrifuging the homogenate from step (i) to obtain a pellet;

(ii) extracting the pellet from step (ii) with a detergent to obtain a detergent soluble fraction and a detergent insoluble fraction; and

(iii) subjecting the detergent soluble fraction to further purification steps.

Anaplasma marginale in the sample may be disrupted by any suitable means such as sonication and/or enzymatic degradation.

Preferably, the detergent is n-dodecyl-N,N-dimethyl-3-ammonio-1-propanesulphonate. Further, it is preferred that the further purification steps include isoelectric focusing.

In another aspect, the present invention provides a fusion protein comprising a polypeptide according to the invention fused to at least one heterologous polypeptide sequence.

Preferably, the at least one heterologous polypeptide sequence is selected from the group consisting of: a polypeptide that enhances the stability of the polypeptide of the present invention, and a polypeptide that assists in the purification of the fusion protein.

In yet another aspect, the present invention provides an isolated polynucleotide, the polynucleotide having a sequence selected from:

(i) a sequence of nucleotides shown in SEQ ID NO:5;

(ii) a sequence of nucleotides shown in SEQ ID NO:6;

(iii) a sequence of nucleotides shown in SEQ ID NO:7;

(iv) a sequence of nucleotides shown in SEQ ID NO:57;

(v) a sequence of nucleotides shown in SEQ ID NO:8;

(vi) a sequence of nucleotides shown in SEQ ID NO:56;

(vii) a sequence encoding a polypeptide according to the present invention;

(viii) a sequence capable of selectively hybridizing to any one of (i) to (iv) under high stringency; and

(ix) a sequence of nucleotides which is at least 50% identical to any one of (i) to (iv),

wherein the polynucleotide encodes a polypeptide that raises an immune response against Ehrlichieae and/or Rickettsieae pathogens when administered to a subject.

Preferably, the polynucleotide is at least 60% identical, more preferably at least 70% identical, more preferably at least 80% identical, more preferably at least 85% identical, more preferably at least 90% identical, more preferably at least 95% identical, and even more preferably at least 99% identical to any one of (i) to (iv).

In a further aspect, the present invention provides a vector comprising at least one polynucleotide of the invention. The vectors may be, for example, plasmid, virus or phage vectors provided with an origin of replication, and preferably a promotor for the expression of the polynucleotide and optionally a regulator of the promotor. The vector may contain one or more selectable markers, for example an ampicillin resistance gene in the case of a bacterial plasmid or a neomycin resistance gene for a mammalian expression vector. The vector may be used in vitro, for example for the production of RNA or used to transfect or transform a host cell. Preferably, the vector is a viral vector.

In another aspect, the present invention provides a host cell comprising a vector of the invention.

Preferably, the host cell is mammalian cell.

In a further aspect, the present invention provides a process for preparing a polypeptide according to the invention, the process comprising cultivating a host cell according to the invention under conditions which allow expression of the polynucleotide encoding the polypeptide, and recovering the expressed polypeptide. This process can be used for the production of commercially useful quantities of the encoded polypeptide.

In a further aspect, the present invention provides a composition comprising a polypeptide according to the invention, and a pharmaceutically acceptable carrier.

The invention will hereinafter be described by way of the following non-limiting Figures and Examples.

### BRIEF DESCRIPTION OF THE DRAWINGS

FIG. 1: Anaplasma marginale protein fractionation strategy outlined in Example 2.

FIG. 2: Anaplasma marginale protein fractionation strategy outlined in Example 3.

FIG. 3: Fractionation of Anaplasma marginale for vaccination trial.

FIG. 4: Fractionation of Anaplasma marginale antigens.

FIG. 5: rAna29 specific antibody titres prior to challenge. Cattle were vaccinated with recombinant protein in CSIRO adjuvant.

FIG. 6: rAna32 antibody titres after vaccination. Cattle were vaccinated with rAna32 in CSIRO adjuvant.

### KEY TO THE SEQUENCE LISTING:

SEQ ID NO:1—Amino acid sequence of Ana 29 from A. marginale.

SEQ ID NO:2—Amino acid sequence of Ana 43 from A. marginale.

SEQ ID NO:3—Amino acid sequence of Ana 37 from A. marginale.

SEQ ID NO:4—Amino acid sequence of Ana 32 from A. marginale.

SEQ ID NO:5—Nucleotide sequence encoding Ana 29 from A. marginale.

SEQ ID NO:6—Nucleotide sequence encoding Ana 43 from A. marginale.

SEQ ID NO:7—Nucleotide sequence encoding Ana 37 from A. marginale.

SEQ ID NO:8—Nucleotide sequence encoding Ana 32 from A. marginale.

SEQ ID NO:9—Fragment from A. marginale Msp-1.

SEQ ID NO:10—Fragment from A. marginale Msp-2.

SEQ ID NO:11—Fragment from A. marginale Msp-4.

SEQ ID NO's:12 to 14—Sequenced fragments of Ana 29 from A. marginale.

SEQ ID NO:15—Sequenced fragment of Ana 32 from A. marginale.

SEQ ID NO's:16 to 20—Sequenced fragments of Ana 37 from A. marginale.

SEQ ID NO's:21 to 26—Sequenced fragments of Ana 43 from A. marginale.

SEQ ID NO's:27 to 55—Oligonucleotide primers used in PCR experiments.

SEQ ID NO:56—Nucleotide sequence of Ana 32 from A. centrale.

SEQ ID NO:57—Nucleotide sequence of Ana 29 from A. centrale.

SEQ ID NO:58 and 59—Fragments of Ana 29 from A. marginale.

### DETAILED DESCRIPTION OF THE INVENTION

Definitions

A “subject” referred to herein is any organism susceptible to a Ehrlichieae or Rickettsieae pathogen. Preferably, the subject is a mammal. Typically, the mammal is selected from the group consisting of cattle, sheep, goats, horses and humans.

By “treating or preventing an Ehrlichieae or Rickettsieae infection in a subject” we mean the reduction or prevention of at least one symptom associated with the infection.

An “immune response” is the total immunological reaction of an animal to an immunogenic stimulus. In general there are considered to be two types of immune responses produced by two populations of lymphocytes. B cells are responsible for humoral immunity, producing antibodies that circulate in the blood stream, whereas T cells are responsible for cell-mediated immunity.

An “immunogen” or an “antigen” is a molecule, typically a polypeptide, that when administered into a subject causes an immune response.

“Anaplasmosis” is characterized by extravascular anaemia associated with intraerythrocytic parasitism. Numerous pathogens are known in the art to cause anaplasmosis, including Anaplasma marginale, Anaplasma centrale and Anaplasma ovis.

Unless stated otherwise, molecular weight values defined herein are as determined by SDS-PAGE.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

General Methods

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 14, IRL Press (1995 and 1996), and F. M. Ausubel et al. (Editors), Current Protocols in Molecular Biology, Greene Pub. Associates and Wiley-Interscience (1988, including all updates until present) and are incorporated herein by reference.

Polypeptides

By “substantially purified polypeptide” we mean a polypeptide that has generally been separated from the lipids, nucleic acids, other polypeptides, and other contaminating molecules with which it is associated in its native state. Preferably, the substantially purified polypeptide is at least 60% free, preferably at least 75% free, and most preferably at least 90% free from other components with which they are naturally associated.

The % identity of a polypeptide is determined by GAP (Needleman and Wunsch, 1970) analysis (GCG program) with a gap creation penalty=5, and a gap extension penalty=0.3. The query sequence is at least 10 amino acids in length, and the GAP analysis aligns the two sequences over a region of at least 10 amino acids. More preferably, the query sequence is at least 20 amino acids in length, and the GAP analysis aligns the two sequences over a region of at least 20 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 200 amino acids in length and the GAP analysis aligns the two sequences over a region of at least 200 amino acids.

It will be appreciated that the present invention encompasses precursors, allelic variants, species homologues, mutants and biologically active fragments of the polypeptides of the invention.

A “precursor” of a polypeptide refers to larger forms of the polypeptide which are processed to produce the polypeptide. This may be achieved through the removal of a hydrophobic signal region from the N-terminus of a precursor. Alternatively, exo- and endopeptidases may cleave precursor molecules to produce a processed polypeptide.

An “allelic variant” will be a variant that is naturally occurring within an individual organism.

Polypeptide sequences are “homologous” or “species homologues” if they are related by divergence from a common ancestor. Consequently, a species homologue of a polypeptide will be the equivalent polypeptide which occurs naturally in another species or strains of a species. Within any one species a homologue may exist as numerous allelic variants, and these will be considered homologues of the polypeptide. Allelic variants and species homologues can be obtained by following standard techniques known to those skilled in the art. Preferred species homologues include those obtained from representatives of the same Order, more preferably the same Family and even more preferably the same Genus.

Amino acid sequence mutants of the polypeptides of the present invention can be prepared by introducing appropriate nucleotide changes into a nucleic acid sequence, or by in vitro synthesis of the desired polypeptide. Such mutants include, for example, deletions, insertions or substitutions of residues within the amino acid sequence. A combination of deletion, insertion and substitution can be made to arrive at the final construct, provided that the final protein product possesses the desired characteristics.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 30 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the antigenic determining region(s), and the active site(s). Other sites of interest are those in which particular residues obtained from various species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

Furthermore, if desired, unnatural amino acids or chemical amino acid analogues can be introduced as a substitution or addition into the polypeptide of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

TABLE 1
Exemplary Substitutions

| Original | Exemplary |
|---|---|
| Residue | Substitutions |
| Ala (A) | val; leu; ile; gly |
| Arg (R) | lys; gln; asn |
| Asn (N) | gln; his; lys; arg |
| Asp (D) | glu |
| Cys (C) | ser |
| Gln (Q) | asn; his |
| Glu (E) | asp |
| Gly (G) | pro; ala |
| His (H) | asn; gln; lys; arg |
| Ile (I) | leu; val; ala; met; phe |
| Leu (L) | ile; val; met; ala; phe |
| Lys (K) | arg; gln; asn |
| Met (M) | leu; phe; ile |
| Phe (F) | leu; val; ala; ile; tyr; trp |
| Pro (P) | gly |
| Ser (S) | thr |
| Thr (T) | ser |
| Trp (W) | tyr; phe |
| Tyr (Y) | trp; phe; thr; ser |
| Val (V) | ile; leu; met; phe; ala |

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Also included within the scope of the invention are biologically active fragments of the polypeptides of the present invention. By “biologically active fragment” we mean a fragment of a sequence of the present invention which retains at least one of the activities of the native polypeptide.

Most preferably, a “biologically active fragment” of the present invention is capable of raising an immune response against a Ehrlichieae or Rickettsieae pathogen when the fragment is administered to a subject. Such fragments are also referred to herein as an “antigenic fragment”. Preferred antigenic fragments include portions of the polypeptides of the present invention which are naturally exposed on the outer surface of the species of Ehrlichieae or Rickettsieae. Examples of such antigenic fragments include, but are not limited to, a polypeptide which has an N-terminal sequence as provided in SEQ ID NO:12 and has a molecular weight of approximately 17 kDa,

| | |
|---|---|
| RLSQEGLESSVLLKRPEFIA, | (SEQ ID NO:58) |
| or | |
| TADLIGSGFAAATPLQQAWV. | (SEQ ID NO:59) |

As would be known to the skilled addressee, techniques for identifying a biologically active fragment or mutant of a polypeptide of the present invention which is capable of raising an immune response against a Ehrlichieae or Rickettsieae pathogen in a subject are well known in the art. For instance, substitutions and/or deletions can be made to the polypeptide of the present invention and the resulting fragment/mutant tested for its ability to raise an immune response against a Ehrlichieae or Rickettsieae pathogen in the subject.

Polypeptides of the present invention can be produced in a variety of ways, including production and recovery of natural proteins, production and recovery of recombinant proteins, and chemical synthesis of the proteins. In one embodiment, an isolated polypeptide of the present invention is produced by culturing a cell capable of expressing the polypeptide under conditions effective to produce the polypeptide, and recovering the polypeptide. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Polynucleotides

By “isolated polynucleotide” we mean a polynucleotide which have generally been separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid molecule”.

The % identity of a polynucleotide is determined by GAP (Needleman and Wunsch, 1981) analysis (GCG program) with a gap creation penalty=5, and a gap extension penalty=0.3. The query sequence is at least 15 nucleotides in length, and the GAP analysis aligns the two sequences over a region of at least 15 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the query sequence is at least 600 nucleotides in length and the GAP analysis aligns the two sequences over a region of at least 600 nucleotides.

A polynucleotide sequence of the present invention may selectively hybridise to a polynucleotide that encodes a polypeptide of the present invention, or a sequence set out in any one of SEQ ID NO's 5 to 8, 56 or 57, under high stringency. Furthermore, oligonucleotides of the present invention have a sequence that hybridizes selectively to a polynucleotide of the present invention. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). It is thus apparent that polynucleotides of the invention can be either naturally occurring or recombinant.

Oligonucleotides of the present invention can be RNA, DNA, or derivatives of either. The minimum size of such oligonucleotides is the size required for the formation of a stable hybrid between an oligonucleotide and a complementary sequence on a nucleic acid molecule of the present invention. The present invention includes oligonucleotides that can be used as, for example, probes to identify nucleic acid molecules, primers to produce nucleic acid molecules or as agents which regulate gene transcription and/or mRNA half-life (e.g., as antisense-, triplex formation-, ribozyme- and/or RNA drug-based reagents).

Vectors

One embodiment of the present invention includes a recombinant vector, which includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

One type of recombinant vector comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, animal, and plant cells. Preferred expression vectors of the present invention can direct gene expression in bacterial, yeast, plant and mammalian cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, plant and mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins).

Recombinant molecules of the present invention may also (a) contain secretory signals (i.e., signal segment nucleic acid sequences) to enable an expressed polypeptide of the present invention to be secreted from the cell that produces the polypeptide and/or (b) contain fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments, as well as natural signal sequences. In addition, a nucleic acid molecule of the present invention can be joined to a fusion segment that directs the encoded protein to the proteosome, such as a ubiquitin fusion segment. Recombinant molecules may also include intervening and/or untranslated sequences surrounding and/or within the nucleic acid sequences of nucleic acid molecules of the present invention.

Host Cells

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells can be either untransformed cells or cells that are already transformed with at least one nucleic acid molecule (e.g., nucleic acid molecules encoding one or more proteins of the present invention). Host cells of the present invention either can be endogenously (i.e., naturally) capable of producing proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite, arthropod, animal and plant cells. Preferred host cells include bacterial, mycobacterial, yeast, plant and mammalian cells. More preferred host cells include Agrobacterium, Salmonella, Escherichia, Bacillus, Listeria, Saccharomyces, Spodoptera, Mycobacteria, Trichoplusia, BHK (baby hamster kidney) cells, MDCK cells (normal dog kidney cell line for canine herpesvirus cultivation), CRFK cells (normal cat kidney cell line for feline herpesvirus cultivation), CV-1 cells (African monkey kidney cell line used, for example, to culture raccoon poxvirus), COS (e.g., COS-7) cells, and Vero cells. Particularly preferred host cells are E. coli, including E. coli K-12 derivatives; Salmonella typhi; Salmonella typhimurium, including attenuated strains; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells.

Recombinant DNA technologies can be used to improve expression of transformed polynucleotide molecules by manipulating, for example, the number of copies of the polynucleotide molecules within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts. The activity of an expressed recombinant protein of the present invention may be improved by fragmenting, modifying, or derivatizing polynucleotide molecules encoding such a protein.

Vaccines

Vaccines may be prepared from one or more polypeptides of the invention. The preparation of vaccines which contain an immunogenic polypeptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with carriers/excipients which are pharmaceutically acceptable and compatible with the active ingredient. Suitable carriers/excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine.

As used herein, the term “adjuvant” means a substance that non-specifically enhances an immune response to an immunogen. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. Further examples of adjuvants include aluminum hydroxide, aluminum phosphate, aluminum potassium sulfate (alum), bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. A preferred adjuvant is the CSIRO adjuvant (which contains 1 mg Quil A, 10 mg DEAE Dextran, 1.2 ml Montanide ISA 50V, and 0.8 ml PBS—per 2 ml). Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably about 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

DNA Vaccines

DNA vaccination involves the direct in vivo introduction of DNA encoding an antigen into cells and/or tissues of a subject for expression of the antigen by the cells of the subject's tissue. Such vaccines are termed herein “DNA vaccines” or “nucleic acid-based vaccines.” Examples of DNA vaccines are described in U.S. Pat. No. 5,939,400, U.S. Pat. No. 6,110,898, WO 95/20660 and WO 93/19183. The ability of directly injected DNA that encodes an antigen to elicit a protective immune response has been demonstrated in numerous experimental systems (see, for example, Conry et al., 1994; Cardoso et al., 1996; Cox et al., 1993; Davis et al., 1993; Sedegah et al., 1994; Montgomery et al., 1993; Ulmer et al., 1993; Wang et al., 1993; Xiang et al., 1994; Yang et al., 1997).

To date, most DNA vaccines in mammalian systems have relied upon viral promoters derived from cytomegalovirus (CMV). These have had good efficiency in both muscle and skin inoculation in a number of mammalian species. A factor known to affect the immune response elicited by DNA immunization is the method of DNA delivery, for example, parenteral routes can yield low rates of gene transfer and produce considerable variability of gene expression (Montgomery et al., 1993). High-velocity inoculation of plasmids, using a gene-gun, enhanced the immune responses of mice (Fynan et al., 1993; Eisenbraun et al., 1993), presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter.

Vaccines Derived from Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Exemplary dicotyledons include corn, tomato, potato, bean, soybean, and the like. Typically the transgenic plant is routinely used as a feed source for farm animals, particularly cows.

Transgenic plants, as defined in the context of the present invention include plants (as well as parts and cells of said plants) and their progeny which have been genetically modified using recombinant DNA techniques to cause or enhance production of at least one polypeptide of the present invention in the desired plant or plant organ.

Several techniques exist for introducing foreign genetic material into a plant cell, and for obtaining plants that stably maintain and express the introduced gene. Such techniques include acceleration of genetic material coated onto microparticles directly into cells (see, for example, U.S. Pat. No. 4,945,050 and U.S. Pat. No. 5,141,131). Plants may be transformed using Agrobacterium technology (see, for example, U.S. Pat. No. 5,177,010, U.S. Pat. No. 5,104,310, U.S. Pat. No. 5,004,863, U.S. Pat. No. 5,159,135). Electroporation technology has also been used to transform plants (see, for example, WO 87/06614, U.S. Pat. Nos. 5,472,869, 5,384,253, WO 92/09696 and WO 93/21335). In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during development and/or differentiation using appropriate techniques described herein.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Examples of plant promoters include, but are not limited to ribulose-1,6-bisphosphate carboxylase small subunit, beta-conglycinin promoter, phaseolin promoter, ADH promoter, heat-shock promoters and tissue specific promoters. Promoters may also contain certain enhancer sequence elements that may improve the transcription efficiency. Typical enhancers include but are not limited to Adh-intron 1 and Adh-intron 6.

Constitutive promoters direct continuous gene expression in all cells types and at all times (e.g., actin, ubiquitin, CaMV 35S). Tissue specific promoters are responsible for gene expression in specific cell or tissue types, such as the leaves or seeds (e.g., zein, oleosin, napin, ACP, globulin and the like) and these promoters may also be used. Promoters may also be active during a certain stage of the plants' development as well as active in plant tissues and organs. Examples of such promoters include but are not limited to pollen-specific, embryo specific, corn silk specific, cotton fiber specific, root specific, seed endosperm specific promoters and the like.

Under certain circumstances it may be desirable to use an inducible promoter. An inducible promoter is responsible for expression of genes in response to a specific signal, such as: physical stimulus (heat shock genes); light (RUBP carboxylase); hormone (Em); metabolites; and stress. Other desirable transcription and translation elements that function in plants may be used.

In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the cauliflower mosaic virus (35S and 19S) and the like may be used.

A number of plant-derived edible vaccines are currently being developed for both animal and human pathogens (Hood and Jilka, 1999). Immune responses have also resulted from oral immunization with transgenic plants producing virus-like particles (VLPs), or chimeric plant viruses displaying antigenic epitopes (Mason et al., 1996; Modelska et al., 1998; Kapustra et al., 1999; Brennan et al., 1999). It has been suggested that the particulate form of these VLPs or chimeric viruses may result in greater stability of the antigen in the stomach, effectively increasing the amount of antigen available for uptake in the gut (Mason et al. 1996, Modelska et al., 1998).

Antibodies

The invention also provides monoclonal or polyclonal antibodies to polypeptides of the invention, or antigenic fragments thereof. Thus, the present invention further provides a process for the production of monoclonal or polyclonal antibodies to polypeptides of the invention.

If polyclonal antibodies are desired, a selected mammal (e.g., mouse, rabbit, goat, cow, etc.) is immunised with an immunogenic polypeptide of the present invention. Serum from the immunised animal is collected and treated according to known procedures. If serum containing polyclonal antibodies to a polypeptide of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention, or antigenic fragments thereof, haptenised to another polypeptide for use as immunogens in animals or humans.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against polypeptides of the present invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in method of detecting polypeptides of the invention present in biological samples by a method which comprises:

(a) providing an antibody of the invention;

(b) incubating a biological sample with said antibody under conditions which allow for the formation of an antibody-antigen complex; and

(c) determining whether an antibody-antigen complex comprising said antibody is formed.

Antibodies of the invention may be bound to a solid support and/or packaged into kits in a suitable container along with suitable reagents, controls, instructions and the like.

### EXAMPLES

### Example 1

### General Techniques

Vaccination and Parasite Challenge

Typically, up to eight (8) groups each of 5 to 7 cattle 4-7 months old and free of the benign haemoparasite Theileria orientalis were randomly allocated into groups by weight as an indication of relative age. Although benign itself, the haemoparasite T. orientalis can convey a variable degree of non-specific immunity against A. marginale. Cattle were vaccinated with A. marginale fractions by subcutaneous injection, twice 4 weeks apart with antigens delivered in Quil A (Biofos 1 mg mL⁻¹) and challenged 2 weeks after the second vaccination with 10⁸ A. marginale-infected erythrocytes (homologous strain) by intravenous injection. On occasion, infections of 10⁶ to 10⁸ parasites were used. Cattle were monitored daily and parasitaemia and haematocrit or PCV decrease determined. Parasitaemia is measured from Giemsa stained thin blood films. Typically, 100 microscope fields are counted and percent parasitaemia calculated by the number of parasites in 100 fields with 300 erythrocytes per field. This method has a sensitivity of 0.003 percent.

Destruction or loss of erythrocytes was measured in experiments described in Example 2 to Example 4 by the decrease in packed cell volume (PCV). In Examples 5, 6 and 9, it was measured by decrease in haematocrit. In both cases, it was reported as percent decrease from pre-challenge. For individual animals an haematocrit or PCV fall of 50% from initial values or a PCV of 15% is the criterion for drug treatment to prevent death. All control animals typically showed symptoms of clinical anaplasmosis.

For vaccination and control groups, the mean parasitaemias were calculated as the geometric means of daily parasitaemias. As another indicator the impact of infection, a cumulative parasitaemia was used, namely the sum of parasitaemias over the peak of infection.

Anaplasma marginale—Parasite Isolation and Membrane Solublisation Procedure

A fraction enriched for Anaplasma marginale was prepared using blood from a donor calf with a parasitaemia preferably in excess of 50%. After removal of plasma and buffy coat the parasites were released by lysis of erythrocytes with 0.83% ammonium chloride (NH₄Cl). Remaining leucocytes and unlysed cells were removed by low speed centrifugation. Parasites and stroma were pelleted by high speed centrifugation and washed exhaustively to remove haemoglobin. After three washes two fairly distinct layers were evident in the pellet. Examination of the two layers by Giemsa stained smears revealed that the lower layer was predominantly parasites whereas the upper layer was erythrocytes stroma with some parasites apparently adhered to the stroma. The lower layer was removed and subjected to Percoll™ gradient centrifugation. This step resulted in the concentration of the parasites into a single broad band. This band was removed and the number of parasites estimated. An aliquot (estimated as 10⁸ organisms) was injected into a naive calf to determine parasite viability. The pre-patency period indicated that although <5% of organisms were viable, the mixture was still infective. This suggests that all essential parasite components are likely to be present in this material.

Typically, three litres of Anaplasma marginale (Gypsy Plains isolate) infected blood was taken from a calf by venipuncture. The blood was collected into heparin. For parasite isolation, the target level of infection was greater than 200 parasites per field at 1,000 times magnification with a PCV preferably at around 20%. The blood was stored at 4° C. until processed (normally 1-2 hr).

Plasma was removed from the blood by centrifugation in a Beckman JA10 rotor (6×500 ml), at 1000 g for 30 min at 4° C. The blood cells were washed three times with cold 50 mM Tris/HCl buffer pH 7.4 containing 0.25 M sucrose. Following thorough mixing of cell and buffer, the mixture was centrifuged as before and the plasma/buffer layer removed. Some of the leucocyte buffy coat was removed at each wash.

Approximately 500 ml of washed blood cells were differentially lysed with the addition of an ammonium chloride solution at a final concentration of 0.75% wt/vol and a final volume of 1.5 L. The mixture was allowed to mix at RT until lysis of the erythrocytes was visible (˜15 min).

Remaining leukocytes and thrombocytes were pelleted at approximately 440 g for 20 min at 4° C. The lysate containing the parasites and erythrocyte ghosts was then subjected to a series of washing steps with cold 25 mM Tris/HCl buffer pH 7.4 containing 0.25 M sucrose, 2 mM EDTA and 50 μM AEBSF. Following each of three wash steps, parasites and erythrocyte ghosts were differentially separated from the lysate/buffer by centrifugation in a Beckman JA14 rotor (6×250 ml), at 17500 g for 30 min at 4° C. On centrifugation, two layers of sediment are formed. The bottom layer contains mainly Anaplasma marginale initial bodies and the top layer predominantly erythrocyte ghosts along with some parasites. At each wash step, a large part of the upper erythrocyte ghosts layer was decanted to waste and the remaining pellet resuspended in fresh buffer. As the volume of parasite containing pellet was reduced, the mixture was transferred to smaller centrifuge tubes (8×40 ml) and centrifuged in a Beckman JA20 rotor, at 18500 g for 20 min at 4° C. (3 washes).

Final separation of the parasites from the remaining erythrocyte ghosts and other cell debri was performed on a 16% Percoll™ gradient. The washed parasite pellet was resuspended in cold 25 mM Tris/HCl buffer pH 7.4 containing 0.25 M sucrose, 2 mM EDTA and 50 μM AEBSF to a volume of 270 ml. A solution of 52.8 ml of Percoll™ and 5.9 ml of 2.5M sucrose was prepared. This solution was then mixed with the parasite solution and loaded into eight 40 ml Beckman Quick-Seal™ tubes. The gradient was formed in situ by centrifugation in a Kontron 65.38 rotor at 20000 g for 1 hr at 4° C. Anaplasma marginale initial bodies aggregate in a loose pellet at the bottom of the tube just above a small Percoll™ pellet. Remaining erythrocyte ghosts and other remaining blood cell components migrate to the upper region of the gradient. The upper region of the gradient was aspirated to waste and the parasite pellet harvested by pipette.

Parasite Membrane Isolation and Solublisation

The parasite material was subjected to sonication with 2×2 minute sonication cycles being sufficient to disrupt the A. marginale initial bodies. Endonuclease (Sigma Cat. No. E8263) was added at 2 Upper ml and allowed to incubate at 4° C. for 30 min. The parasite material was then centrifuged in a Kontron 65.38 rotor at 100,000 g for 1 hr at 4° C. The parasite membrane pellet was collected and resuspended in 50 mM Tris/HCl pH 7.4 containing 2 mM EDTA and 50 μM AEBSF (Tris/HCl⁺⁺) and recentrifuged as above. The membrane pellet was resuspended in Tris/HCl⁺⁺ to a volume of 12 ml.

The membrane material was combined with 12 ml of 4% (wt/vol) n-dodecyl-N,N-dimethyl-3-ammonio-1-propanesulphonate (purchased as Zwittergent® 3-12 from Boehringer Mannheim) in Tris/HCl⁺⁺ and incubated at RT, with rocking, for 2 hr. Following incubation the material was centrifuged in a Beckman 70.1 TI rotor at 100,000 g for 1 hr at 4° C. The supernatant was collected and named the Zwittergent® 3-12 soluble material.

Isoelectric Focusing (IEF)

The Zwittergent® 3-12 soluble material was then prepared for wide-range isoelectricfocusing (WR-IEF). To 100 ml of Milli Q water (MQW), 18 ml of Ampholine™ pH 3.5-10.0 (Amersham Pharmacia Biotech, Uppsala) and 1.35 g Zwittergent® 3-12 were added and dissolved. The volume was adjusted to 270 ml with MQW and 16.0 g Ultrodex™ granulated gel (Amersham Pharmacia Biotech, Uppsala) added and allowed to swell fully. The Multiphor II IEF system (Amersham Pharmacia Biotech, Uppsala) was used for the IEF. A flat bed gel was prepared (19 cm×24 cm×0.5 cm deep) and the IEF gel pre-focused at 12 W and 10° C. for 1500 Vh. Meanwhile, to 19.0 ml of the Zwittergent® 3-12 soluble material, 1.36 ml Ampholine™ pH 3.5-10.0 and approximately 1.0 g Ultrodex™ granulated gel were combined and the gel allowed to swell fully. The sample was loaded into the prefocused IEF gel at the low pH end (region of final pH ˜4.0-4.3). The Zwittergent® 3-12 soluble material was focused at 12 W and 10° C. for 12000 Vh.

Following electrophoresis, the IEF slab gel was cut horizontally into 30 equal fractions, giving fractions from low pH to high pH. The aqueous portion was separated from the granulated gel by centrifugation through a gauze sieve. The gel was washed (2×2 ml) with MQW containing 0.2% Zwittergent® 3-12 and 50 μM AEBSF. The pH of each fraction was recorded and the sample pH adjusted to 7.4 with 1.0 ml of 1.0M Tris-HCl pH 7.4 buffer.

The protein components of the IEF fractions were visualised on SDS-PAGE.

Western Blotting using Antisera Raised to Conjugated Synthetic Peptides from known Antigen Sequences

Removal of Previously Characterized Antigens

MSP-1 and related proteins including AmV70. A probe was required to enable this protein to be detected and its elimination from vaccine fractions monitored. Rabbit antisera have been produced to a peptide derived from analysis of the amino acid sequence (15 amino acids CQRVAAQERSRELSRA (SEQ ID NO:9)). A cysteine residue was included to facilitate conjugation to keyhole limpet haemocyanin. The anti-MSP-1 peptide antibody proved to be a useful reagent in the identification of MSP-1 by Western blotting of SDS-PAGE gels, under reducing conditions.

MSP-2 and MSP-4. Additional rabbit antisera were raised against a number of parasite polypeptides, initially to be used for protein identification. These include: CVDGHINPKFAYRVK (SEQ ID NO:10) from MSP 2, and ESSKETSYVRGYDKSIATIDC (SEQ ID NO:11) from MSP 4.

Antisera Raised to Purified Native Anaplasma Antigens, Whose Identity Had Been Confirmed by N-Terminal Sequencing

MSP-2 and MSP4 were purified using a number of chromatography techniques and identity confirmed by N-terminal amino acid sequence. Polyvalent antisera were produced in rabbits using standard techniques. Antisera prepared against MSP-2 specifically recognised only MSP-2 but antisera to MSP-4 recognised both MSP-2 and MSP4. Examination of the database showed sufficient sequence homologue to explain this result. We concluded that these probes will be satisfactory reagents to determine the presence or absence of these proteins in fractions prepared from A. marginale.

### Example 2

### Demonstration that Soluble, Membrane Bound and Detergent Extractable Material from Anaplasma marginale Contains Protective Antigens

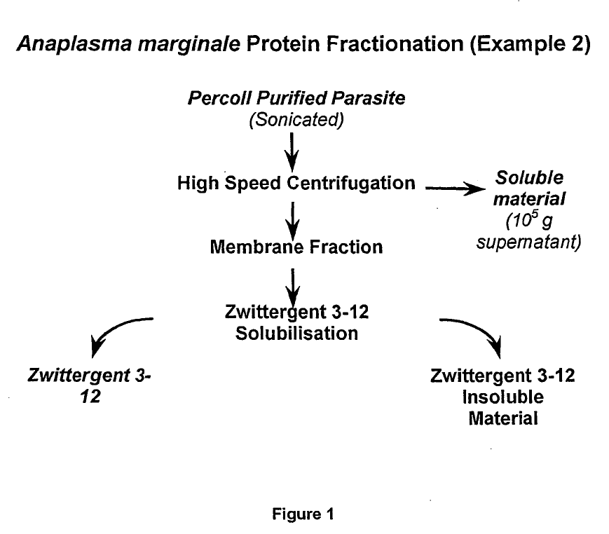

A fraction of parasite crude antigen was prepared by sonication of purified parasites while an equivalent fraction was prepared using blood from the donor calf collected prior to infection with A. marginale. Ultracentrifugation (100,000 g) gave a soluble fraction. The pellet was extracted with Zwittergent®3-12 for 90 minutes at 37° C. and ultracentrifuged (100,000 g) to give detergent soluble and detergent insoluble fractions. All fractions were analysed by SDS-PAGE under reducing conditions. SDS-PAGE showed that the majority of the host material had been removed as indicated by the absence of major bovine proteins. The sub-fractions of the starting material showed that there were bands common between fractions, particularly between the detergent soluble and insoluble materials, though there were also unique species in each fraction. The procedure is shown in FIG. 1.

Vaccination efficacy was defined as those fraction(s) which showed a statistically significant difference in parasitaemia compared with the Control Group (calves vaccinated with normal erythrocyte material).

Significant protection was induced by vaccination with 3-12 solubilized material and the residue after 3-12 extraction, particularly when compared with the starting whole parasite extract. The results are shown in Table 2.

TABLE 2
Results of vaccination with complex Anaplasma antigens

| | | Max parasit. | Day max. | | No. cattle |
|---|---|---|---|---|---|
| No. | Group | (%) | parasit. | CP^{#} | treated* |
| 1 | Whole parasite | 7.1 | 18 | 32.1 | 2/7 |
| 2 | Soluble material | 7.2 | 18 | 28.1 | 1/7 |
| 3 | 3-12 soluble | 1.9 | 16 | 10.3 | 1/7 |
| 4 | 3-12 precipitate | 0.5 | 22 | 2.8 | 0/7 |
| 5 | Control | 3.8 | 20 | 19.5 | 2/7 |

*No. cattle treated is the number treated by day 23, when the experiment was terminated
^{#}CP is cumulative parasitaemia, the mean daily parasitaemia for the group, summed over days 12 to 22 (n = 11).

Neither the whole parasite material nor the soluble material induced protection: in fact, they appeared in this experiment to exacerbate the parasitaemia. This shows again the variability experienced in vaccination with crude material, as well as the ability of an inappropriate immune response to exacerbate the disease.

### Example 3

### Fractionation of Detergent Soluble Material from Anaplasma marginale by IEF

Parasite Isolation and Protein Fractionation for Vaccination Trial

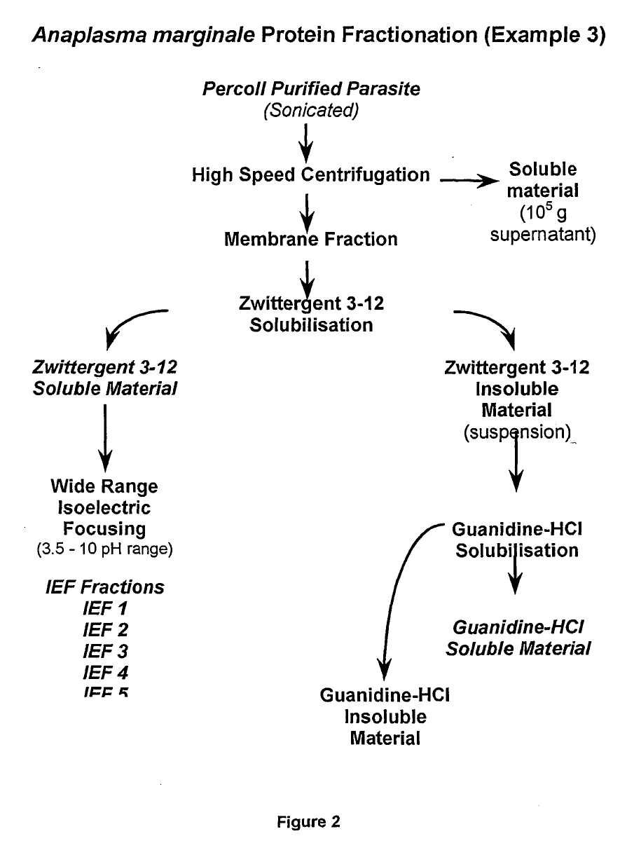

A. marginale parasites were isolated as previously described in General Techniques. Proteins were fractionated to the Zwittergent® 3-12 soluble and 3-12 insoluble step, as described in General Techniques. Further fractionation of parasite proteins was achieved by running the Zwittergent® 3-12 soluble fraction on a flat bed wide-range IEF (3.5-10) as described in General Techniques and the IEF fractions pooled into groups. Five IEF groups were tested in the vaccination/challenge experiment. In addition, the Zwittergent® 3-12 insoluble material was treated with 6M guanidine-HCl and the soluble proteins released by this treatment were also tested. The process is shown in FIG. 2.

Freshly isolated parasites were used for the preparation of material for the 2^{nd} vaccination. The fractionation procedure was easily repeated with the protein profiles of corresponding vaccination fractions exhibiting good reproducibility when compared with the fractions used for the first vaccination. The fractionation procedure is robust and reproducible.

Three (3) A. marginale major surface proteins (MSPs) previously identified and sequenced have been identified in our protein fractions by N-terminal sequencing (MSP-1 “Am 105”, MSP-2 and MSP-4). In addition rabbit anti-peptide antibodies to MSP-1 were used to identify MSP-1. The occurrence of the known MSPs in vaccination fractions is given in Table 3.

TABLE 3
Anaplasma marginale vaccination groups

| Group | Antigen | Major Known Species |
|---|---|---|
| 1 | IEF Fraction 1 pI 6.8-8.6 | Hb α-chain |
| 2 | IEF Fraction 2 pI 5.7-6.8 | MSP 2, Hb α-chain |
| 3 | IEF fraction 3 pI 5.2-5.7 | MSP 2, MSP 4 |
| 4 | IEF Fraction 4 pI 4.2-5.2 | Am 105 |
| 5 | IEF Fraction 5 pI 3.1-4.2 | |
| 6 | Guanidine-HCl Soluble Material | |
| 7 | Total Zwittergent ® 3-12 Soluble | |
| 8 | Control | |

Antigens to be characterized further (the Ana antigens) were also visible in some of the vaccination fractions. Because of their low abundance however, some fractions almost certainly contained one or more of the Ana antigens, though at levels that could not be distinguished in complex protein mixtures.

Vaccination/Challenge Experiment

Eight groups (Table 3) each of 6 cattle 5-7 months old were vaccinated with A. marginale fractions and challenged as described in General Techniques. Cattle were monitored for parasitaemia and haematocrit decrease (Table 4). All control animals (Group 8) showed symptoms of clinical anaplasmosis (maximum parasitaemia >10%) and severe decline in PCV. Two animals required treatment. All treatment groups showed some degree of control of parasitaemia. One animal in Groups 1, 2, 6 and 7 required treatment, using as criteria for treatment a drop in PCV to 0.15 L L⁻¹.

TABLE 4
Vaccination results

| | | | | Max. | Day | | |
|---|---|---|---|---|---|---|---|
| | | Max | | PCV | max. | | No. |
| | | parasit. | Day max. | decline | PCV | | cattle |
| No. | Group | (%) | parasit. | (%) | decline | CP^{#} | treated |
| 1 | IEF 1 | 2.6 | 16 | 42 | 19 | 8.9 | 1/6 |
| 2 | IEF 2 | 3.4 | 16 | 44 | 18 | 11.3 | 1/6 |
| 3 | IEF 3 | 2.3 | 18 | 41 | 20 | 8.7 | 0/6 |
| 4 | IEF 4 | 0.8 | 18 | 34 | 19 | 2.9 | 0/6 |
| 5 | IEF 5 | 2.5 | 18 | 36 | 21 | 7.9 | 0/6 |
| 6 | GuHCL | 5.0 | 18 | 39 | 21 | 13.9 | 0/5 |
| 7 | 3-12 | 6.9 | 16 | 51 | 19 | 22.9 | 1/6 |
| 8 | Control | 16.4 | 17 | 53 | 19 | 64.6 | 2/6 |

^{#}CP is cumulative parasitaemia, the sum of the daily mean parasitaemias from days 14 to 19 inclusive (n = 5).

The parasitaemia data for this experiment showed that all IEF Fractions demonstrated a degree of efficacy in controlling parasitaemia. However, the PCV data demonstrates that cattle vaccinated with IEF Fractions 4 and 5 and guanidine-HCl soluble material had a smaller decrease compared with controls and other groups. This suggests that vaccination with more defined fractions of A. marginale may result in an immune response which controls parasitaemia but does not result in a serious decrease in PCV.

These results therefore demonstrate that A. marginale Zwittergent® 3-12 soluble material can be fractionated into 5 fractions using preparative isoelectric focusing (IEF), all of which fractions showed a significant degree of efficacy. The Guanidine-HCl solubilised material also demonstrated some degree of efficacy. Some IEF fractions contained MSP antigens (viz MSP-1, MSP-2 and MSP4). These were identified by N-terminal amino acid sequence. Others contained no detectable/identifiable previously known MSPs.

### Example 4

### Further Fractionation of IEF Purified Antigens and Separation from Previously Identified Antigens

Although the polyvalent antisera against MSP-1, MSP-2 and MSP4 referred to above were satisfactory for the detection of these proteins on Western blots, they were not suitable for the quantitative removal of these antigens from purifications fractions. A variety of procedures were therefore tested on partially purified Anaplasma material for their ability to remove these antigens. High pressure ion exchange and size exclusion chromatography under a variety of conditions were unable to achieve the necessary resolution. Hydroxyapatite HPLC was found to be useful. An array of dye ligand affinity supports were also tested. Finally, a combination of IEF, hydroxyapatite and dye ligand methods gave the necessary fractions.

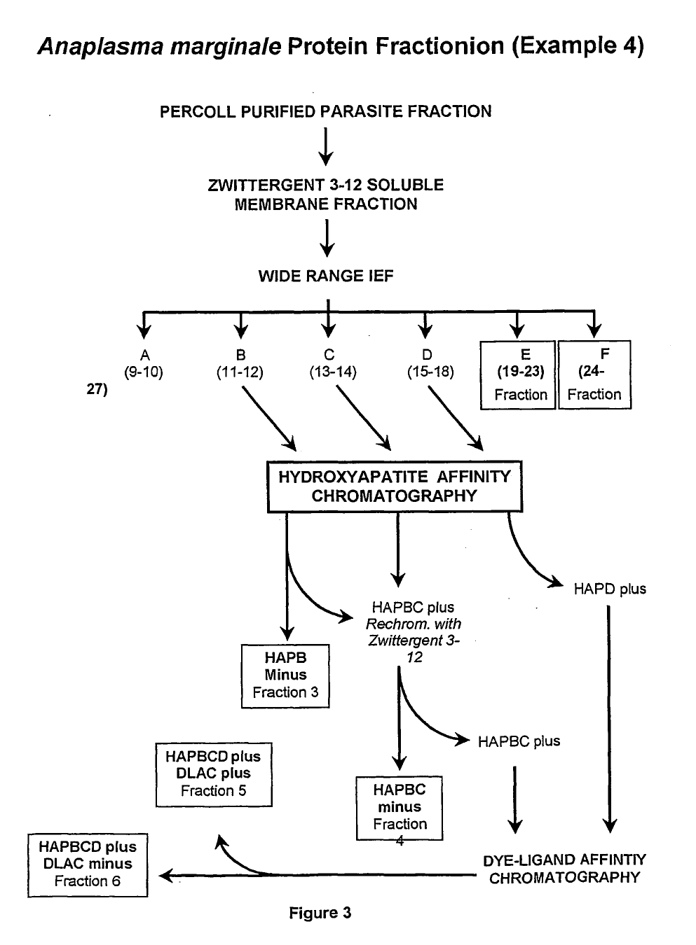

Briefly, freshly isolated parasites were used for the preparation of material for this vaccination experiment. Parasite material was solubilised in Zwittergent® 3-12 and subjected to wide range isoelectrofocussing (IEF) as described in General Techniques. The fractionation procedure was easily repeated with the protein profiles of corresponding vaccination fractions exhibiting good reproducibility when compared with the fractions used for the two previous vaccination experiments. A number of these IEF fractions were subjected to hydroxyapatite chromatography and the unbound material was then subjected to dye ligand chromatography. A total of 6 fractions were generated three of which were shown to contain either MSP-2 or MSP4, while MSP-1, 2 and 4 could not be detected in the other three fractions. The procedure is summarized in FIG. 3, while Table 5 lists the fractions. Antigens characterized further in this report were visible in some of the vaccination fractions. Because of their low abundance however, some fractions almost certainly contained one or more of the Ana antigens, though at levels that could not be distinguished in complex protein mixtures. The identifiable antigens in each of the vaccination fractions are listed in Table 7 as are the occurrence of the previously identified MSPs.

Vaccination Results

A vaccination/challenge experiment was carried out using these six (6) fractions (Table 5). The results are shown in Table 6.

TABLE 5
Vaccination Fractions Example 4

| Vaccination | |
|---|---|
| Group | Protein Fraction |
| 1 | WR-IEF (1) Pool E |
| 2 | WR-IEF (1) Pool F |
| 3 | HAP B minus |
| 4 | HAP BC minus |
| 5 | HAP BCD plus, DLAC plus |
| 6 | HAP BCD plus, DLAC minus |
| 7 | Normal Saline |

All protein fractions showed some effect in reducing erythrocyte destruction and parasitaemia. Some animals needed to be treated in each of Groups 4, 5, 6 and 7 (see Table 6). Groups 4&5, containing predominantly MSP4 and MSP2 respectively, induced the poorest protection of all protein fractions. Groups 1 &2 produced the best protective immune response with the parasitaemia remaining at low levels, resulting in a significant reduction in erythrocyte destruction. The fact that fractions 1 &2 contained no detectable MSPs is strong evidence that there are novel protective antigens in Anaplasma marginale. The protein profile of fractions 1 &2 (data not shown) shows a number of protein species common to both fractions. The possibility that the same protective antigen/s are present in both fractions is apparent.

TABLE 6
Vaccination results

| | | | | Max. | Day | | |
|---|---|---|---|---|---|---|---|
| | | Max | Day | PCV | max. | | No. |
| | | parasit. | max. | decline | PCV | | cattle |
| No. | Group | (%) | parasit. | (%) | decline | CP^{#} | treated |
| 1 | IEF (1) E | 3.2 | 19 | 37 | 21 | 15.9 | 0/5 |
| 2 | IEF (1) F | 3.6 | 17 | 39 | 19 | 18.9 | 0/5 |
| 3 | HAPB − | 11.6 | 19 | 49 | 22 | 41.2 | 0/5 |
| 4 | HAPBC − | 11.8 | 18 | 43 | 19 | 46.9 | 2/5 |
| 5 | HAP | 7.7 | 18 | 43 | 24 | 47.0 | 3/5 |
| | BCD +/+ | | | | | | |
| 6 | HAP | 7.1 | 17 | 43 | 20 | 31.8 | 1/5 |
| | BCD +/− | | | | | | |
| 7 | Saline | 25.7 | 17 | 52 | 19 | 98.9 | 2/5 |

^{#}CP is cumulative parasitaemia, the sum of the daily mean parasitaemias from days 12 to 21 inclusive (n = 9).

TABLE 7
Major proteins visible in vaccination fractions

| Group | Major novel species | Major known species |
|---|---|---|
| IEF (1) E | Ana 29, Ana 17, two major | Hb α-chain |
| | unknown proteins | |
| IEF (1) F | Ana 17 | Hb α-chain |
| HAPB − | Complex proteins | MSP 4 |
| HAPBC − | 30 kDa protein | MSP 4 |
| HAP BCD +/+ | None | MSP 2, Hb α-chain |
| HAP BCD +/− | Complex proteins | None |

### Example 5

### Further Fractionation of Antigens in the PI Range 7.7 to 9.5

In Example 4, the major MSPs were separated from other less abundant proteins resulting in several vaccination fractions which contained specific groups of MSP-free proteins. The isolated MSP proteins were also assessed. These induced only low levels of protection. The MSP-free fractions induced varying levels of protection. The two wide-range IEF fractions, IEF-E and IEF-F (incorporating proteins of pI 7.30-9.07) were the most efficacious on parasite challenge.

MSP2 in particular is present in very large amounts in parasite extracts. This, plus its hypervariable nature, make its removal from fractions prior to vaccination trials an ongoing challenge.

Success in antigen fractionation and purification has the inevitable consequence that isolating enough material for a vaccination trial becomes an ever increasing difficulty. In Example 4, the combined IEF fractions E & F (IEF-EF) equated to less than 1% of the total parasite protein (348 mg). This 1% was the “starting material” for the current example. The IEF-EF fraction contains seven or eight major proteins and a considerable number (>15) of less abundant species. Accordingly, even the major proteins in this fraction are minor species in the A. marginale organism as a whole. Nevertheless, the IEF-EF fraction proteins have been separated further and proteins recovered in adequate quantities. Fractionation of A. marginale proteins with isoelectric points greater than approximately 7.7 was the main focus of Example 5.

Fractionation of Proteins in the IEF-EF Fraction (pI 7.7 to 9.5)

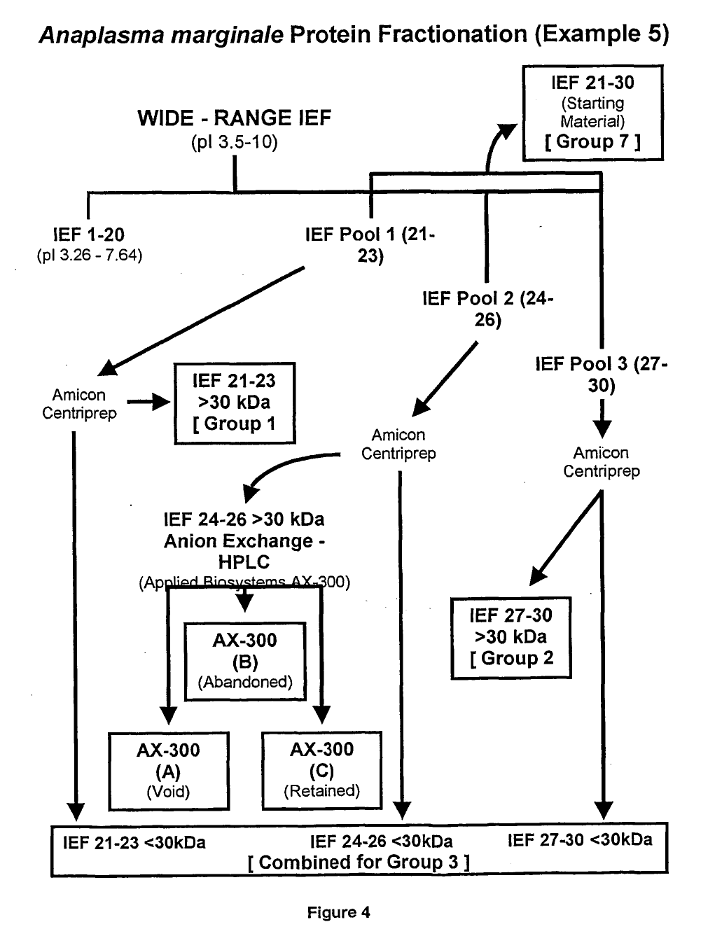

Fractionation of potential antigens from the IEF-EF pool was designed to segregate the most abundant protein species. Some of these proteins were present in both IEF-E and IEF-F protective fractions in Example 4. Major proteins between 17 and 57 kDa were selected as “target” species, to be separated from each other. Proteins were isolated using a number of techniques including: (a) IEF (b) ultrafiltration through a 30 kDa cut-off filter (mainly to isolate low molecular weight material [<10 kDa] prevalent in the high pI fractions) and (c) anion exchange (AE)-HPLC on an Applied Biosystems AX-300 anion exchanger. A flow diagram of the fractionation procedure is shown in FIG. 4. The major protein species in each fraction are listed in Table 8.

Vaccination Trial

Owing to the low abundance of “target” proteins in the IEF-EF fraction, the blood from three infected splenectomized calves was needed for parasite isolation. Nevertheless, the amount of protein per vaccination per animal was still relatively low (Table 9). Even the most abundant species in the vaccination trials will be present only in low microgram amounts. Since the nature of the protective immune response has been only partially characterised, our ability to elicit the appropriate response can be gauged only through parasite challenge.

TABLE 8
Major proteins visible in vaccination fractions

| | | Major known |
|---|---|---|
| Group | Major novel species | species |
| IEF 21-23 >30 KDA | Ana 29, Ana 17 | Hb α-chain |
| IEF 27-30 >30 kDa | Ana 43, Ana 32 | Hb α-chain |
| IEF 21-30 <30 kDa | | None |
| AX-300 (A) [void] | Ana 43, Ana 37, | Hb α-chain |
| | Ana 32, Ana 17 | |
| AX-300 (B) | | None |
| [retained] | | |
| AX-300 (C) | Ana 29 | None |
| [retained] | | |
| IEF 21-30 [starting | All the above | None |
| material] | | |

TABLE 9
Vaccination fractions - protein estimations

| | | pI Range of | Protein/animal/ |
|---|---|---|---|
| Group | | source | vacc. |
| Number | FRACTION | material | (μg) |
| 1 | IEF 21-23 >30 kDa | 7.83-8.10 | 23 |
| 2 | IEF 27-30 >30 kDa | 8.99-9.55 | 17 |
| 3 | IEF 21-30 <30 kDa | 7.83-9.55 | 12 |
| 4 | AX-300 (A) [void] | 8.28-8.71 | 13 |
| 5 | AX-300 (B) [retained] | 8.28-8.71 | not measurable |
| 6 | AX-300 (C) [retained] | 8.28-8.71 | 6 |
| 7 | IEF 21-30 [starting material] | 7.83-9.55 | 36 |

Vaccination results are given in Table 10. Of all the vaccination groups, Fraction 1 (IEF 21-23>30 kDa) was clearly the most protective, demonstrating the least erythrocyte destruction (17% fall in Hct, day 17) and the lowest parasitaemia (5-fold reduction relative to controls). The starting material, Group 7 (equivalent to IEF-EF, the protective fraction of Example 4) also showed a significant reduction in erythrocyte destruction and in parasitaemia (31% of controls, day 17) confirming the previous result. The control group and non-protected vaccinated groups showed reductions in Hct on day 17 of 39% to 45%.

TABLE 10
Vaccination results

| | | | | Max. | Day | | |
|---|---|---|---|---|---|---|---|
| | | Max | Day | Hct | max. | | No. |
| | | parasit. | max. | decline | Hct | | cattle |
| No. | Group | (%) | parasit. | (%) | decline | CP^{#} | treat. |
| 1 | IEF 21-23 | 2.5 | 19 | 41 | 24 | 12.2 | 1/5 |
| | >30 KDA | | | | | | |
| 2 | IEF 27-30 | 5.6 | 16 | 43 | 24 | 34.9 | 1/5 |
| | >30 kDa | | | | | | |
| 3 | IEF 21-30 | 14.2 | 15 | 53 | 19 | 75.7 | 3/5 |
| | <30 kDa | | | | | | |
| 4 | AX-300 (A) | 7.5 | 17 | 51 | 24 | 35.8 | 3/5 |
| | [void] | | | | | | |
| 5 | AX-300 (B) | 9.3 | 15 | 50 | 19 | 61.7 | 4/5 |
| | [retained] | | | | | | |
| 6 | AX-300 (C) | 7.9 | 15 | 45 | 19 | 43.0 | 0/5 |
| | [retained] | | | | | | |
| 7 | IEF 21-30 | 4.1 | 18 | 45 | 24 | 22.3 | 2/5 |
| | [starting | | | | | | |
| | material] | | | | | | |
| 8 | Controls | 11.2 | 18 | 54 | 21 | 74.7 | 3/5 |

^{#}CP is cumulative parasitaemia, the sum of the daily mean parasitaemias from days 12 to 20 inclusive (n = 9).

In conclusion, proteins in the pI range 7.7 to 9.5 were fractionated using a combination of ultrafiltration and anion exchange high performance liquid chromatography (AE-HPLC). Six distinct groups of proteins were isolated in sufficient quantity for a vaccination trial in cattle. The best protected group was group 1, with groups 2, 4 and 6 also showing significant protection, measured by a reduction in parasitaemia and increased survival.

Group 1 antigen was relatively complex, the major protein species being Ana 29 and Ana 17 (where, for example, Ana 29 indicates that the polypeptide has a MW of about 29 kDa). Group 2 contained principally Ana 43 and Ana 32. Group 4 was again complex, containing as the major species proteins probably identifiable with Ana 43, 37, 32 and 17. Group 6 contained principally Ana 29 with some minor species.

### Example 6

### Identification of Individual Protective Antigens

Example 5 tested the efficacy of fractions separated from the IEF-EF pool, the most efficacious fraction of Example 4. “Target” proteins were isolated using a number of techniques. In summary, Fraction 1, containing predominantly Ana 29, Ana 17 and bovine Hb alpha chain was clearly the most protective, demonstrating the least erythrocyte destruction (17% reduction in Hct, day 17) and the lowest parasitaemia (5-fold reduction relative to controls). Other fractions, containing varying amounts and combinations of Ana 43, Ana 37 Ana 32 and Ana 29, showed protection, but at a lower level. The starting material, Group 7 (equivalent to IEF-EF, the protective fraction of Example 5) also showed a significant reduction in erythrocyte destruction and parasitaemia (31% of controls, day 17) confirming the previous result. The control group and non-protected vaccinated groups showed reductions in Hct on day 17 of 39% to 45%.

The protein Ana 29 and a protein migrating close to 25 kDa were subjected to N-terminal sequencing several times and found to possess identical N-terminal sequence. It is likely that the 25 kDa protein may be a partial breakdown product of Ana 29. Therefore, both the 25 kDa and the 29 kDa form are referred to as Ana 29. Both have been present in vaccination trials.

Isolation of Novel Protective Anaplasma marginale Antigens

SDS-PAGE Isolation of Proteins in the WR-IEF pI 7.8-9.8 Fraction

Three splenectomised steers were infected with Anaplasma marginale and used for the collection of parasites. Anaplasma marginale parasites were isolated and fractionated using the standard protocol.

The major A. marginale proteins present in the WR-IEF pI range 7.8 to 9.8 fraction were purified to homogeneity on SDS-PAGE. The “target” proteins for isolation in this instance, included not only the major proteins present in Fraction 1 as outlined in Example 5 (the most efficacious), but those present in less efficacious but still protective fractions. “Target” proteins included: Ana 43, Ana 37, Ana 32, Ana 29 and Ana 17. Protein bands resolved on SDS PAGE were visualised with Coomassie Brilliant Blue R-250, excised from the gel, passively eluted with an acetate/SDS/DTT buffer, then methanol precipitated to remove undesirable detergent and acrylamide residues.

Vaccination Trial

The five proteins nominated above were purified in sufficient quantities and of purity suitable for vaccination. These proteins were Ana 43, Ana 37, Ana 32, Ana 29 and Ana 17. Other proteins in the SDS-PAGE gel (between 17 kDa and 100 kDa), present in only minor amounts were also extracted from the gel, pooled together and represent the sixth protein fraction (nominated “Rest”). Estimates of the amount of protein injected into each animal at each of the two vaccinations are shown in Table 11. It is noted that the amount of Ana 43, and Ana 32 injected in the trial is low and is a direct result of the difficulty of precipitating these species in methanol in the detergent removal step. Evidence of the efficacy of these two proteins together was obtained in Example 5. The amounts of Ana 29 and Ana 17 injected in this experiment are more than in Example 5. The results of the vaccination trial are shown in Table 11.

Identification of Proteins by N-Terminal Sequencing

Approximately a fifth of the protein material (WR-IEF pI 7.8-9.8) fractionated from the first two donor animals was used for the acquisition of N-terminal amino acid sequence data. Various WR-IEF pI 7.8-9.8 fractions were run on SDS-PAGE then blotted to PVDF membrane. Protein bands were excised and sequenced using an Applied Biosystems 471A Protein Sequencer. The result of the N-terminal amino acid sequencing is shown in Table 12. Ana 43, Ana 37 and Ana 29 possessed novel sequence. No significant sequence homology to these proteins was obtained from the protein sequence databases (Swiss-Prot and EMBL). As previously known, a major protein present in the WR-IEF pI 7.9-9.8 fraction, was sequenced and confirmed to be bovine Hb alpha chain. Since this work was performed, a gene sequence which, when translated, has an N-terminal sequence identical to that of Ana 32 has been reported in the literature (Barbet et al., 2000).

TABLE 11
Vaccination results

| | | | | | | Max. | | |
|---|---|---|---|---|---|---|---|---|
| | | Protein/ | Max | Day | | Hct | Day | |
| | | animal/ | para- | max. | | de- | max. | No. |
| | | vaccn. | sit. | para- | | cline | Hct | cattle |
| No. | Group | (μg) | (%) | sit. | CP^{#} | (%) | decl. | treat. |
| 1 | Ana 43 | <2.0* | 9.7 | 18 | 65 | 46 | 21 | 1/5 |
| 2 | Ana 37 | 4.1* | 11.4 | 16 | 60 | 45 | 21 | 2/5 |
| 3 | Ana 32 | <2.0* | 6.9 | 16 | 46 | 42 | 23 | 0/5 |
| 4 | Ana 29 | 26.3* | 9.1 | 15 | 56 | 44 | 21 | 1/6 |
| | | (27.7**) | | | | | | |
| 5 | Ana 17 | 10.0* | 6.4 | 19 | 59 | 44 | 23 | 0/5 |
| 6 | “Rest” | 44.4** | 7.1 | 18 | 47 | 38 | 21 | 1/5 |
| | >16 kDa, | | | | | | | |
| | <100 kDa | | | | | | | |
| 7 | Controls | | 17.8 | 17 | 98 | 46 | 21 | 1/6 |

^{#}CP is cumulative parasitaemia: the sum of the daily mean parasitaemias from days 13 to 24 inclusive (n = 12).
Protein concentrations presented (*) were estimated from silver stained bands on SDS-PAGE gels. Anaplasma protein bands were compared to the carbonic anhydrase band (0.83 μg protein/μl of standard loaded).
Protein amounts (**) were estimated using the Pierce BCA reagent with BSA as the standard.

Ana29, Ana37 and Ana43 have been further sequenced and comprise the sequences shown in Table 12. In addition, it has been established that Ana 17 is a C-terminally truncated antigenic fragment of Ana 29.

Furthermore, the complete open reading frame of the gene encoding Ana 32 has been sequenced from an Australian isolate of A. marginale (SEQ ID NO:8). The protein encoded by this open reading frame is provided in SEQ ID NO:4.

TABLE 12
Partial Ana Sequences

| | |
|---|---|
| Ana29 | SFKIKDERLSAHIANPDGTRYMRQG |
| | (SEQ ID NO:12) |
| Ana2901 | HVSFVS/RSGR (SEQ ID NO:13) |
| Ana29P105 | E/GMN/EGLYPNAAYLVAPA |
| | (SEQ ID NO:14) |
| Ana32 | AAPNVGSAAPGVGAEGEL |
| | (SEQ ID NO:15) |
| Ana37 | SPRPIDES/QRGEGASGFFASVQYK |
| | (SEQ ID NO:16) |
| Ana37_A | VGVQYFASR (SEQ ID NO:17) |
| Ana37_B | AAL/IFAYAYASR (SEQ ID NO:18) |
| Ana37_C | GPDL/IASGGSFEGK (SEQ ID NO:19) |
| Ana37_D | QSVSVGYSEL/IVR (SEQ ID NO:20) |
| Ana43 | AEAFGPYVSFGYTPAAGDV |
| | (SEQ ID NO:21) |
| Ana43H10/18 | LNLXN/FLFTAT (SEQ ID NO:22) |
| Ana43H5/12 | PYLSYSDK (SEQ ID NO:23) |
| Ana43H04 | HTMLGQALPK (SEQ ID NO:24) |
| Ana43H04b | ASVFVGGVLHR (SEQ ID NO:25) |
| Ana43H14P12 | I/LXPYGVTGAV (SEQ ID NO:26) |

### Example 7

### Cloning and Characterization of the cDNA Encoding Ana29, Ana32, Ana37 and Ana43

Parasite RNA Extraction and cDNA Synthesis

Total RNA was prepared from 10 ml A. marginale (Gypsy Plains isolate) or Anaplasma centrale infected blood using Trizol Reagent as per manufacturer's recommendations (Life Technologies). For cDNA synthesis, 2 μg of total RNA was reverse transcribed in a 20 μl reaction mix using 0.5 μg/μl oligo (dT)₁₂₋₁₈ primer and Superscript II (Life Technologies) at 42° C. for 70 min.

For 5′ RACE and 3′ RACE cDNA synthesis, approximately 2 μg of total RNA was reverse transcribed in a 20 μl reaction using 1 μM of SMART oligo II, 1 μM oligo dT and Powerscript Reverse Transcriptase (Clontech Laboratories Inc., Palo Alto, Calif.) at 42° C. for 70 min.

RT-PCR Reactions

A standard PCR reaction consists of 45 mM Tris-HCl (pH 8.8), 11 mM NH₄SO₄, 4.5 mM MgCl₂, 6.7 mM 2-mercaptoethanol, 4.4 μM EDTA (pH8.0), 1 mM each of the four dNTP's and 1 μM of each oligonucleotide primer. 0.5-1 μl of cDNA prepared as above was used in all PCR reactions.

Degenerate oligonucleotides were designed to the N-terminal and internal protein sequences of Ana29, Ana37 and Ana43 as follows: Ana25-2,5′ GACGGNACNAGRTAYATG (SEQ ID NO:27); Ana25-3,5′ GGATANARNCCYTCCATYTC (SEQ ID NO:28); Ana37-C, 5′CGCTWGCGTAWGCRTANGC (SEQ ID NO:29); Ana37-E 5′ TTCTTCGCWAGYGTNCARTAYM (SEQ ID NO:30); Ana43-A, 5′GCTGARGCNTTYGGNCCNTAYGT (SEQ ID NO:31) and Ana43-B 5′ACTGCBCCWGTNACNCCRTANGG (SEQ ID NO:32). The cycling conditions used were as follows: denature 94° C. for 20 s, anneal 40° C. for 40 s, extension at 72° C. for 2 min. The annealing temperature was increased by 0.5° C. for 20 cycles, followed by 10 cycles at 50° C. For Ana43, additional sequence was obtained using the primers Ana43-H 5′TGATGAAGTATTATCCTGGTCTAGCT (SEQ ID NO:33) and the degenerate primer Ana43-C₅′ GCYTGNCCNAGCATNGTRTG (SEQ ID NO:34).

For 5′ RACE and 3′RACE PCR, cycling conditions used were 94° C. for 20 s, 70° C. for 40 s and 72° C. for 2 min for 10 cycles, followed by 10 cycles at 65° C. annealing then a further 15 cycles at 60° C. Primers used for 5′ RACE were Ana25-5 5′ TAGCGCTGATGGTCATGCTG (SEQ ID NO:35) and Ana25-6, 5′ CGTGGTCTGCAATGAACATCAG (for nested PCR) (SEQ ID NO:36); Ana37-J 5′TCGGACGAGCCCGCTGGAGGCGTT (SEQ ID NO:37); Ana37-K 5′ AAGCGCCTTACCTTTGTCTTCTACTA (nested PCR) (SEQ ID NO:38); Ana43-E 5′CAGGACATACCAAGTCTCTCCAGGA (SEQ ID NO:39) and Ana43-F 5′CMCGTATAGGTTTCTAACACCTC (nested PCR) (SEQ ID NO:40). For 3′ RACE the following primers were used: Ana25-7,5′ CTGACGCATATACCTCGTAC (SEQ ID NO:41) and Ana25-8, 5′GGCAACTACTATGACCGAC (nested PCR) (SEQ ID NO:42), Ana37-L 5′GCGGTGGTGGCAGTCTAGTCAGGT (SEQ ID NO:43); Ana37-M 5′GGTGCAGTATTTCGCGTCTAGGM (nested PCR) (SEQ ID NO:44); Ana43-J2 5′TMCTATGTTGGGTCGGCGACCATG (SEQ ID NO:45) and Ana43-K 5′CCGGCAGCAACTGGTCAAGTAAGC (nested PCR) (SEQ ID NO:46). Gene specific primers were used in conjunction with universal and nested universal primers as supplied by Clontech Laboratories Inc. (Palo Alto, Calif.).

Amplification of the full-length Ana29 gene fragment was obtained using primers Ana25-F, 5′GAGGATCCATGTCCTTCAAGATTA (SEQ ID NO:47) and Ana25-R1, 5′TACCCGGGTTACATTGCAACGGGAGTT (SEQ ID NO:48). Full length Ana32, Ana37 and Ana43 were amplified using the following primer sets: Ana32-B 5′TGGATCCGGCGGCTCCCTTGT (SEQ ID NO:49), Ana32-C 5′ACCCGGGTCAAAGTTTCACCCTTATG (SEQ ID NO:50); Ana37-N 5′GCATGCTCCCCCAGGCCCATAGACT (SEQ ID NO:51), Ana37-P 5′ CCCGGGATAAGTACGAGTCTTATNCCG (SEQ ID NO:52); Ana43-M 5′GGATCCGCCGAAGCATTTGGTCCGTAC (SEQ ID NO:53), Ana43-R 5′GTCGACCTACGTGAGTATAAGCCTCA (SEQ ID NO:54). Restriction sites were included in the 5′ ends of the primers to enable sub-cloning of the PCR fragment into expression vectors. PCR cycle conditions used were 94° C. for 20 s, 50° C. for 40 s, 72° C. for 1 min for 30 cycles.

The homologues of A. marginale Ana 29 and Ana 32 were also obtained from A. centrale. Primers used to amplify the genes were Ana25-F (SEQ ID NO:47) and Ana25-R1 (SEQ ID NO:48) for Ana29; Ana32-A 5′TGGATCCATGAGTCGTAAAAGTCTG (SEQ ID NO: 55) and Ana32-C (SEQ ID NO:50). PCR cycling conditions used were 94° C. for 30 s, 50° C. for 40 s and 72° C. for 1 min repeated 35 times.

20 μl of the PCR reaction was electrophoresed through a 1% agarose gel in 1×TAE buffer and visualised with ethidium bromide. Bands were excised from the gel and the DNA recovered by centrifugation through a spin column (Sigma-Aldrich) for 10 min at 10 000 g.

Standard procedures were used for gel electrophoresis, sub-cloning, and transformation and growing of E. coli (Sambrook et al., 1989).

Cloning and Sequencing

The eluted PCR products were cloned into pCR2.1 TA vector (Invitrogen, California) following manufacturer recommendations. Plasmid DNA from at least two recombinant colonies were isolated using a plasmid mini-prep kit (QIAGEN Inc., California). The insert size was determined by comparing EcoRI digestion of the plasmid to a 1 kb DNA ladder after electrophoresis.

Full-length genes were excised from pCRAna25 F/R1 and pCRAna32 B/C by digestion with BamHI and SmaI followed by agarose gel separation. pCRAna43 MIR was digested with BamHI and SalI to recover the full length gene. Restriction enzymes SphI and SmaI were used to subclone pCRAna37 P/N. The recovered fragments were ligated into pQE30 vector digested with appropriate enzymes (QIAGEN Inc., California). Recombinants were analysed for the presence of the insert and designated as pQEAna254, pQEAna32 B/C, pQEAna37N/P and pQEAna43 M/R for plasmids encoding the A. marginale proteins Ana29, Ana32, Ana37 and Ana43 respectively. These plasmids were used to transform E. coli Ml 5 pRep 4 strain for production of recombinant proteins.

Plasmids containing inserts were sequenced using Big Dye terminator chemistry on an ABI Prism 377 DNA sequencer (Perkin-Elmer Applied Biosystems, California). DNA sequence and deduced amino acid sequences were analysed using the Wisconsin GCG software package (Genetic Computer Group, Inc., Madison, Wis.). DNA and deduced amino acid sequences were compared to sequences in public databases by BLAST at NCBI, Sanger and TIGR comprehensive microbial resource. No significant homologies were found in sequence databases using Ana29 DNA or amino acid sequences, limited homology (approx 30%) to known A. marginale MSP 4 and Ehrlichial outer membrane proteins were obtained with Ana32, Ana37 and Ana43 amino acid sequences.

The Ana32 sequence (SEQ ID NO:8) contains an open reading frame of 894 bp encoding for 297 amino acids (SEQ ID NO:4). The theoretical MW of this protein is 31 kDa and has a pI of 9.32. The protein contains a signal peptide of 23 amino acids, which was omitted from pQEAna32 B/C. Four nucleotide substitutions, at position's 119, 267, 303 and 808 bp, were observed between Ana32 from the Gypsy Plains, Australian isolate compared with the published sequence from American isolates (Barbet et al., 2000). These code for two changes in the amino acid sequence (99% identity). Furthermore, the homologous polynucleotide sequence from A. centrale (SEQ ID NO:56) was found to contain only one silent base change at position 504 bp when compared to SEQ ID NO:8. Thus, the Ana 32 polypeptide sequence from A. centrale is identical to that disclosed herein from A. marginale (SEQ ID NO:4).

The Ana29 polynucleotide sequence (SEQ ID NO:5) contains an open reading frame of 786 bp encoding for 261 amino acids (SEQ ID NO:1). All the native Ana29 peptide sequences listed in Table 12 are found within the translated cDNA sequence. The theoretical MW of this protein is 27 kDa and has a pI of 8.6. The homologous polynucleotide sequence from A. centrale (SEQ ID NO:57) was found to contain only two silent base changes at positions 294 bp and 669 bp when compared to SEQ ID NO:5. Thus, the Ana 29 polypeptide sequence from A. centrale is identical to that disclosed herein from A. marginale (SEQ ID NO:1).

The Ana43 polynucleotide sequence (SEQ ID NO:6) contains an open reading frame of 1269 bp encoding for 422 amino acids (SEQ ID NO:2). The theoretical MW of this protein is 45 kDa and has a pI of 9.2. Internal peptide sequences obtained (Table 12) are represented in the translated sequence. No initiating methionine codons were found upstream of the known N-terminal hence the start of the gene is unknown. However an in frame stop codon was found 48 amino acids upstream of the mature protein N-terminal, suggesting the presence of a signal peptide up to 48 amino acids in length.

The Ana37 polynucleotide sequence (SEQ ID NO:7) contains an open reading frame of 990 bp encoding for 330 amino acids (SEQ ID NO:3). The theoretical MW of this protein is 35 kDa and has a pI of 8.2. All internal peptide sequences obtained (Table 12) are represented in the translated sequence.

Sequence analysis indicates that Ana 32, Ana 37 and Ana 43 are all surface proteins. Ana 29 possesses four potential trans membrane regions, and hence may function as a transporter.

### Example 8

### Expression and Purification of Recombinant Proteins rAna29, rAna32, rAna37 AND rAna43

The recombinant A. marginale proteins, pQEAna254, pQEAna32 B/C, pQEAna43 MIR and pQEAna37 NIP were expressed in E. coli M15 pRep4 cells and purified by affinity chromatography on nickel-nitrilo-tri-acetic acid resin as described in the Qiagen protocol.

SDS-PAGE gradient gels (Laemmli, 1970) stained with silver nitrate (Morrissey, 1981) were used to visualise protein expression and degree of purity. For Western blots, proteins were blotted onto Hybond C (Amersham) and the recombinant protein detected with Ni-Nta-HRP (Qiagen). Colour development was with 4-chloro-napthol. The recombinant protein concentrations were determined after purification with the Pierce BCA reagent.

The yield obtained from a one-litre culture of E. coli expressing the Ana29 gene was 2.5 mg of purified rAna29. After long-term storage at 4° C. a degree of aggregation of the recombinant protein was observed. Similar yields of purified recombinant protein were obtained from one litre cultures of E. coil for rAna32 (3.8 mg) and rAna43 (8.5 mg). No aggregation of these proteins was observed after storage at 4° C.

### Example 9

### Vaccination/Challenge Experiments with Recombinant Antigens

Groups of 6 cattle, 5-7 months old, were vaccinated using a total of 100 μg of purified recombinant protein in two doses. In all vaccination experiments cattle were challenged 2 weeks after the final vaccination with 108 erythrocytes infected with A. marginale. Cattle were monitored daily for parasitaemia and haematocrit drop. The results are shown in Table 13. Control groups of cattle in Vaccination 2001-1 and 2001-2 were injected with 100 μg of an irrelevant recombinant protein in the CSIRO adjuvant. Control groups of cattle in Vaccination 2001-3 were vaccinated with adjuvant only. Anaplasma challenge of Vaccination 2001-1 and 2001-2 were performed concurrently.

In a 2 ml dose per animal the CSIRO adjuvant contained 1 mg Quil A, 10 mg DEAE Dextran, 1.2 ml Montanide ISA 50V, and 0.8 ml PBS.

Vaccination 2001-1

For Vaccination 2001-1, Ana29 recombinant protein was injected at time 0 and 6 weeks using CSIRO adjuvant.

Ana29 recombinant protein in the CSIRO adjuvant significantly protected cattle against challenge-(Group 2 Table 13). Importantly no cattle in this group required drug treatment, a significantly lower parasitaemia (5.1±4.4%) and a smaller decrease in haematocrit (33.4%) were observed in comparison with controls (12±6.6% and 42.2% respectively, P<0.001) (Group 1 Table 13). All control animals showed symptoms of clinical anaplasmosis and a severe decline in haematocrits.

Vaccination 2001-2

Cattle in this trial received two vaccinations with rAna32 in the CSIRO adjuvant separated by 3 weeks (Group 4 Table 13). In this vaccination some protection was observed as a reduced haematocrit drop and a delay in peak parasitaemia in comparison to the controls (Group 3 Table 13). Fewer cattle required treatment in vaccinated versus control cattle.

Vaccination 2001-3

Cattle received two vaccinations with rAna43 in adjuvant 4 weeks apart. Vaccinated cattle were significantly protected (P<0.01) against challenge with A. marginale as demonstrated by the reduced parasitaemia (11.8 compared with 25.5 for the controls) and a smaller and delayed decrease in the haematocrits (Group 6 cf. Group 5 Table 13). All control animals showed symptoms of clinical anaplasmosis and a severe decline in haematocrits with all six animals in the control group requiring treatment compared with only one animal in the vaccinated group.

TABLE 13
Vaccination 2001-1, 2 & 3

| | | | | Max. | Day | | |
|---|---|---|---|---|---|---|---|
| | | Max. | Day | PCV | Max. | | No. |
| Group | | Parasit. | Max. | decline | PCV | | cattle |
| No | Antigen | (%) | parasit. | (%) | decline | CP# | treated |
| 1 | Control | 12 | 16 | 42 | 19 | 71.3 | 1/6 |
| 2 | rAna29 | 5.1 | 18 | 33 | 19 | 37.5 | 0/6 |
| 3 | Control | 10.3 | 15 | 50 | 21 | 60.1 | 3/6 |
| 4 | rAna32 | 11.3 | 18 | 45 | 20 | 76.2 | 1/6 |
| 5 | Control | 25.5 | 19 | 61 | 21 | 97.2 | 6/6 |
| 6 | rAna43 | 11.8 | 19 | 47 | 27 | 45.7 | 1/6 |

# CP is cumulative parasitaemia, the sum of the daily mean parasitaemia's from days 13 to 24 inclusive for groups 1-4, days 14 to 21 inclusive for groups 5 and 6.

Antibody Analysis by ELISA

Immunolon 2 microtitre plates (Dynex) were coated with 1 μg/ml rAna29, rAna32 or rAna43 in carbonate buffer. All washes were performed with PBS/Tween (0.1%). Test sera were initially diluted 1:1000 followed by doubling dilutions across the plate. A control positive and control negative sera, consisting of a mix of four sera from vaccinated and control cattle, was included on each plate to standardise assays. Sheep anti-bovine IgG, IgG1 and IgG2 HRP secondary antibodies were obtained from Serotec and used at 1:3000 dilutions. Colour was developed by the addition of ABTS substrate and read on a Mulktiskan Ascent microtitre plate reader at 405 nm. Titres were determined at an arbitrarily chosen OD and standardised to the positive control sera. Antibody titres were estimated by extrapolating the linear section of plots of optical density against log (inverse dilution).

Vaccination 2001-1

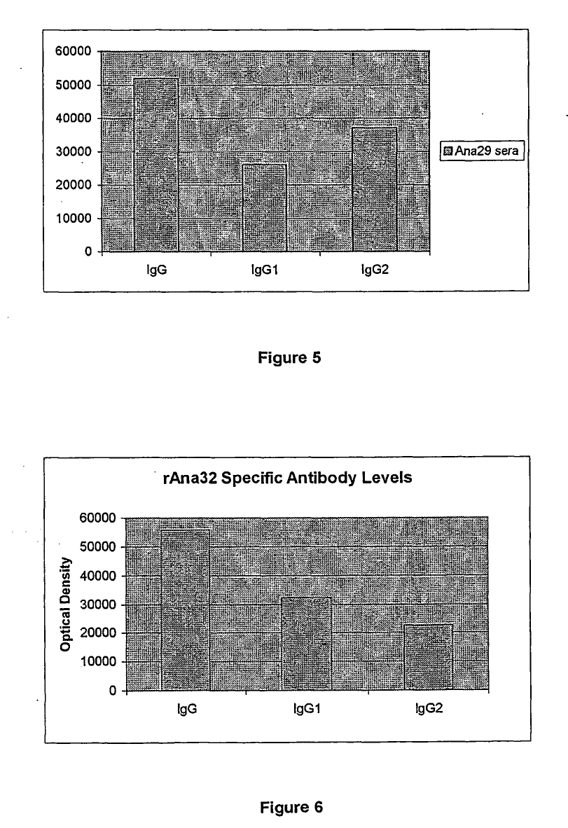

Antibody levels were determined for cattle vaccinated with rAna29 in adjuvant. The titers for rAna29 specific IgG, IgG1 and IgG2 after vaccination were 52000, 26000 and 37000 respectively (FIG. 5). The ratio of IgG2:IgG was 0.7, suggesting IgG2 antibodies represent approximately 70% of total IgG antibodies present in the vaccinated cattle sera.

Vaccination 2001-2

The titres for rAna32 specific IgG, IgG1 and IgG2 were 56000, 32000 and 22500 respectively (FIG. 6). IgG2 comprises approximately 40% of the total IgG specific to rAna32 stimulated by vaccination.

Vaccination 2001-3

Total rANA43 specific IgG, IgG1 and IgG2 titres were 27400, 12000 and 10000 respectively. IgG2 comprises approximately 36% of total IgG specific to rAna43 stimulated by vaccination, similar to that observed in Vacc2001-2.

In conclusion we have demonstrated a significant degree of protection is observed after vaccination using the recombinant proteins rAna29, rAna43 and r Ana32. We have also shown that significant amounts of antibody are raised against the specific recombinant proteins after vaccination.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### REFERENCES

Barbet, A. F., Lundgren, A., Yi, J., Rurangirwa, F. R. and Palmer, G. H. (2000) Infection and Immunity 68: 6133-6138.
Bock, R. E., de Vos, A. J., Kingston, T. G. and McLellan, D. J. (1997) Australian Veterinary Journal 75: 337-340.
Brennan F. R., Bellaby T., Helliwell S. M., Jones T. D., Kamstrup S., Dalsgaard K., Flock J-I. and Hamilton W. D. O. (1999) Journal of Virology 73:930-938.
Cardoso A. I., Blixenkrone-Moller M., Fayolle J., Liu M., Buckland R. and Wild T. F. (1996) Immuniz Virol 225:293-299.
Conry, R. M., LoBuglio, A. F., Kantor, J., Schlom, J., Loechel, F., Moore, S. E., Sumerel, L. A., Barlow, D. L., Abrams, S. and Curiel, D. T. (1994) Cancer Research 54: 1164-1168.
Cox, G. J., Zamb, T. J. and Babiuk, L. A. (1993) Journal of Virology 67:5664-5667.
Davis, H. L., Michel, M. L. and Whalen, R. G. (1993) Human Molecular Genetics 2:1847-1851.
Eisenbraun, M. D., Fuller, D. H. and Haynes, J. R. (1993) DNA and Cell Biology 12:791-797.
Fynan, E. F., Webster, R. G., Fuller, D. H., Haynes, J. R., Santoro, J. C. and Robinson, H. L. (1993) Proc Natl Acad Sci USA 90:11478-11482.
Hood, E. E. and Jilka, J. M. (1999). Current Opinions in Biotechnology 10:382-386.
Kapustra J., Modelska A., Figlerowicz M., Pniewski T., Letellier M., Lisowa O., Yusibov V., Koprowski H., Plucienniczak A. and Legocki A. B. (1999) FASEB J 13:1796-1799.
Laemmli, E. K. (1970) Nature 227:680-685.
Mason H. S., Ball J. M., Shi J. J., Jiang X., Estes M. K and Arntzen C. J. (1996) Proc Natl Acad Sci USA 93:5335-5340.
Modelska A., Dietzschold B., Sleysh N., Fu Z. F., Steplewski K., Hooper D. C., Koprowski H. and Yusibov V. (1998) Proc Natl Acad Sci USA 95:2481-2485.
Montenegro-James, S., James, M. A., Toro Benitez, M., Leon, E., Baek, B. K. and Guillen, A. T. (1991) Parasitology Research 77: 93-101.
Montgomery, D. L., Shiver, J. W., Leander, K. R., Perry, H. C., Friedman, A., Martinez, D., Ulmer, J. B., Donnelly, J. J. and Lui, M. A. (1993) DNA and Cell Biology 12:777-783.
Morrissey, J. H. (1981) Anal. Biochem. 117:307-310.
Needleman, S. B. and Wunsch, C. D. (1970) Molecular Biology 48; 443-453.
Palmer, G. H., Barbet, A. F., Davis, W. C. and McGuire, T. C. (1986) Science USA 231: 1299-1302.
Sambrook, J., Fritsch, E. F. and Maniatis, T. (1989) Molecular Cloning: A Laboratory Manual. 2nd Edition. Cold Spring Harbour Laboratory Press, USA.
Sedegah, M., Hedstrom, R., Hobart, P. and Hoffman, S. L. (1994) Proc Natl Acad Sci USA 91:9866-9870.
Ulmer, J. B., Donnelly, J. J., Parker, S. E., Rhodes, G. H., Feigner, P. L., Dwarki, V. J., Gromkowski, S. H., Deck, R. R., DeWitt, C. M., Friedman, A. et al. (1993) Science 259:1745-1749.
Vidotto, M. C., McGuire, T. C., McElwain, T. F., Palmer, G. H. and Knowles, D. P. Jr. (1994) Infection and Immunity 62: 2940-2946.
Wang, B., Ugen, K. E., Srikantan, V., Agadjanyan, M. G., Dang, K., Refaeli, Y., Sato, A. I., Boyer, J., Williams, W. V. and Weiner, D. B. (1993) Proc Natl Acad Sci USA 90:4156-4160.
Xiang, Z. Q., Spitalnik S., Tran, M., Wunner, W. H., Cheng, J. and Ertl, H. C. (1994) Virology 199:132-140.
Yang K., Mustafa F., Valsamakis A., Santoro J. C., Griffin D. E. and Robinson H. L. (1997) Vaccine 15:888-891.

1. A vaccine comprising at least one polypeptide selected from the group consisting of:
a) a sequence provided in SEQ ID NO:1;
b) a polypeptide which is at least 80% identical to (a);
c) a sequence provided in SEQ ID NO:2;
d) a polypeptide which is at least 80% identical to (c);
e) a sequence provided in SEQ ID NO:3;
f) a polypeptide which is at least 80% identical to (e);
g) a sequence provided in SEQ ID NO:4; and
h) a polypeptide which is at least 80% identical to (g);
wherein the polypeptide raises an immune response against Ehrlichieae and/or Rickettsieae pathogens when administered to a subject.

2.-4. (canceled)

5. The vaccine according to claim 1, wherein the vaccine comprises a pharmaceutically acceptable carrier.

6. The vaccine according to claim 1, wherein the vaccine comprises an adjuvant.

7. A DNA vaccine comprising at least one polynucleotide selected from the group consisting of:
a) a sequence encoding a polypeptide provided in SEQ ID NO:1;
b) a sequence encoding a polypeptide which is at least 80% identical to SEQ ID NO:1;
c) a sequence encoding a polypeptide provided in SEQ ID NO:2;
d) a sequence encoding a polypeptide which is at least 80% identical to SEQ ID NO:2;
e) a sequence encoding a polypeptide provided in SEQ ID NO:3;
f) a sequence encoding a polypeptide which is at least 80% identical to SEQ ID NO:3;
g) a sequence encoding a polypeptide provided in SEQ ID NO:4; and
h) a sequence encoding a polypeptide which is at least 80% identical to SEQ ID NO:4;
wherein the polypeptide encoded by the polynucleotide raises an immune response against Ehrlichieae and/or Rickettsieae pathogens when the DNA vaccine is administered to a subject.

8.-10. (canceled)

11. The DNA vaccine according to claim 7, wherein the vaccine comprises a pharmaceutically acceptable carrier.

12. The DNA vaccine according to claim 7, wherein the polynucleotide is contained in a vector.

13. (canceled)

14. A method for raising an immune response against an Ehrlichieae or Rickettsieae pathogen in a subject, the method comprising administering to the subject at least one vaccine according to claim 1.

15. A method of treating or preventing an Ehrlichieae or Rickettsieae infection in a subject, the method comprising administering to the subject at least one vaccine according to claim 1.

16.-17. (canceled)

18. The method according to claim 14, wherein the subject is a mammal selected from the group consisting of; cows, sheep, goats, dogs and horses.

19.-20. (canceled)

21. A transgenic plant which produces at least one polypeptide selected from the group consisting of:
a) a sequence provided in SEQ ID NO:1;
b) a polypeptide which is at least 80% identical to (a);
c) a sequence provided in SEQ ID NO:2;
d) a polypeptide which is at least 80% identical to (c);
e) a sequence provided in SEQ ID NO:3;
f) a polypeptide which is at least 80% identical to (e);
g) a sequence provided in SEQ ID NO:4; and
h) a polypeptide which is at least 80% identical to (g);
wherein the polypeptide raises an immune response against Ehrlichieae and/or Rickettsieae pathogens when the transgenic plant is orally administered to a subject.

22. A method for raising an immune response against an Ehrlichieae or Rickettsieae pathogen in a subject, the method comprising orally administering to the subject at least one transgenic plant according to claim 21.

23. A method of treating or preventing an Ehrlichieae or Rickettsieae infection in a subject, the method comprising orally administering to the subject at least one transgenic plant according to claim 21.

24. An antibody raised against a polypeptide selected from the group consisting of:
a) a sequence provided in SEQ ID NO:1;
b) a polypeptide which is at least 80% identical to (a);
c) a sequence provided in SEQ ID NO:2;
d) a polypeptide which is at least 80% identical to (c);
e) a sequence provided in SEQ ID NO:3;
f) a polypeptide which is at least 80% identical to (e);
g) a sequence provided in SEQ ID NO:4; and
h) a polypeptide which is at least 80% identical to (g);
wherein the antibody provides immune response against Ehrlichieae and/or Rickettsieae pathogens when administered to a subject.

25. A method of treating or preventing an Ehrlichieae or Rickettsieae infection in a subject, the method comprising administering to the subject at least one antibody according to claim 24.

26. A substantially purified polypeptide which specifically binds to an antibody according to claim 24.

27. A substantially purified polypeptide, the polypeptide being selected from:
(i) a polypeptide comprising the sequence provided as SEQ ID NO:1; and
(ii) a polypeptide which is at least 80% identical to (i);
wherein the polypeptide raises an immune response against Ehrlichieae and/or Rickettsieae pathogens when administered to a subject.

28. (canceled)

29. A substantially purified polypeptide, the polypeptide being selected from:
(i) a polypeptide comprising the sequence provided as SEQ ID NO:2; and
(ii) a polypeptide which is at least 80% identical to (i);
wherein the polypeptide raises an immune response against Ehrlichieae and/or Rickettsieae pathogens when administered to a subject.

30. A substantially purified polypeptide, the polypeptide being selected from:
(i) a polypeptide comprising the sequence provided as SEQ ID NO:3; and
(ii) a polypeptide which is at least 80% identical to (i);
wherein the polypeptide raises an immune response against Ehrlichieae and/or Rickettsieae pathogens when administered to a subject.

31. A substantially purified polypeptide, the polypeptide being selected from:
(i) a polypeptide comprising the sequence provided as SEQ ID NO:4; and
(ii) an antigenic fragment of (i),
wherein the polypeptide, or antigenic fragment thereof, raises an immune response against Ehrlichieae and/or Rickettsieae pathogens when administered to a subject.

32.-34. (canceled)

35. A fusion protein comprising a polypeptide according to claim 27 fused to at least one heterologous polypeptide sequence.

36. An isolated polynucleotide, the polynucleotide having a sequence selected from:
(i) a sequence of nucleotides shown in SEQ ID NO:5;
(ii) a sequence of nucleotides shown in SEQ ID NO:6;
(iii) a sequence of nucleotides shown in SEQ ID NO:7;
(iv) a sequence of nucleotides shown in SEQ ID NO:57;
(v) a sequence of nucleotides shown in SEQ ID NO:8;
(vi) a sequence of nucleotides shown in SEQ ID NO:56;
(vii) a sequence encoding a polypeptide according to claim 16;
(viii) a sequence capable of selectively hybridizing to any one of (i) to (iv) under high stringency; and
(ix) a sequence of nucleotides which is at least 80% identical to any one of (i) to (iv),
wherein the polynucleotide encodes a polypeptide that raises an immune response against Ehrlichieae and/or Rickettsieae pathogens when administered to a subject.

37. A vector comprising at least one polynucleotide according to claim 36.

38. (canceled)

39. A host cell comprising the vector of claim 37.

40. (canceled)

41. A process for preparing a polypeptide according to claim 27, the process comprising cultivating a host cell according to claim 39 under conditions which allow expression of the polynucleotide encoding the polypeptide, and recovering the expressed polypeptide.

42. (canceled)
